SEQUENCE LISTING (29 sequences)

SEQ ID NO: 1 - DNA, 20 bp, Artificial Sequence
  Description of Artificial Sequence Synthetic primer
atccacctgccttagtccac

SEQ ID NO: 2 - DNA, 20 bp, Artificial Sequence
  Description of Artificial Sequence Synthetic primer
tcgaagaactgttccagcca

SEQ ID NO: 3 - DNA, 19 bp, Artificial Sequence
  Description of Artificial Sequence Synthetic primer
gaatcctgcgcaccgagat

SEQ ID NO: 4 - DNA, 22 bp, Artificial Sequence
  Description of Artificial Sequence Synthetic primer
ctgcacttgaaccctgcaatac

SEQ ID NO: 5 - DNA, 23 bp, Artificial Sequence
  Description of Artificial Sequence Synthetic primer
cagcgagattctcccaagctaag

SEQ ID NO: 6 - DNA, 22 bp, Artificial Sequence
  Description of Artificial Sequence Synthetic primer
aaccctaaccacatgagcaacg

SEQ ID NO: 7 - DNA, 23 bp, Artificial Sequence
  Description of Artificial Sequence Synthetic primer
ggaaagcaaaagcccctctgaat

SEQ ID NO: 8 - DNA, 21 bp, Artificial Sequence
  Description of Artificial Sequence Synthetic primer
accctcaccctcaccctaagc

SEQ ID NO: 9 - DNA, 23 bp, Artificial Sequence
  Description of Artificial Sequence Synthetic primer
cagcaagtgggaaggtgtaatcc

SEQ ID NO: 10 - DNA, 25 bp, Artificial Sequence
  Description of Artificial Sequence Synthetic primer
cccattctatcatcaacgggtacaa

SEQ ID NO: 11 - DNA, 37 bp, Artificial Sequence
  Description of Artificial Sequence Synthetic primer
ggtttttgagggtgagggtgagggtgagggtgagggt

SEQ ID NO: 12 - DNA, 39 bp, Artificial Sequence
  Description of Artificial Sequence Synthetic primer
tcccgactatccctatccctatccctatccctatcccta

SEQ ID NO: 13 - DNA, 18 bp, Artificial Sequence
  Description of Artificial Sequence Synthetic probe
  misc_feature (1)..(1): 5′ tagged with Alexa546
ttagggttagggttaggg

SEQ ID NO: 14 - DNA, 18 bp, Artificial Sequence
  Description of Artificial Sequence Synthetic probe
  misc_feature (1)..(1): 5′ tagged with Alexa488-OO
ccctaaccctaaccctaa

SEQ ID NO: 15 - DNA, 37 bp, Artificial Sequence; identical to SEQ ID NO: 11
  Description of Artificial Sequence Synthetic primer

SEQ ID NO: 16 - DNA, 39 bp, Artificial Sequence; identical to SEQ ID NO: 12
  Description of Artificial Sequence Synthetic primer

SEQ ID NO: 17 - DNA, 18 bp, Artificial Sequence
  Description of Artificial Sequence Synthetic probe
  misc_feature (1)..(1): 5′ tagged with AF488-OO
ccctaaccctaaccctaa

SEQ ID NO: 18 - DNA, 18 bp, Artificial Sequence
  Description of Artificial Sequence Synthetic probe
  misc_feature (18)..(18): 3′ tagged with AlexF594N
ttagggttagggttaggg

SEQ ID NO: 19 - DNA, 18 bp, Artificial Sequence
  Description of Artificial Sequence Synthetic probe
  misc_feature (18)..(18): 3′ biotin labeled
ccctaaccctaaccctaa

SEQ ID NO: 20 - DNA, 21 bp, Artificial Sequence
  Description of Artificial Sequence Synthetic oligonucleotide
  Description of Combined DNA/RNA Molecule Synthetic oligonucleotide
cccuaaucaucauccaggatt

SEQ ID NO: 21 - DNA, 45 bp, Artificial Sequence
  Description of Artificial Sequence Synthetic oligonucleotide
gagaacggtcctaggcctgtcaagaggagaacggtcctaggcctg

SEQ ID NO: 22 - DNA, 20 bp, Artificial Sequence
  Description of Artificial Sequence Synthetic primer
atccacctgccttagtccac

SEQ ID NO: 23 - DNA, 20 bp, Artificial Sequence
  Description of Artificial Sequence Synthetic primer
tcgaagaactgttccagcca

SEQ ID NO: 24 - DNA, 25 bp, Artificial Sequence
  Description of Artificial Sequence Synthetic primer
cccattctatcatcaacgggtacaa

SEQ ID NO: 25 - DNA, 45 bp, Artificial Sequence
  Description of Artificial Sequence Synthetic oligonucleotide
gatatcagacctacgggtgtcaagaggatatcagacctacgggtg

SEQ ID NO: 26 - DNA, 45 bp, Artificial Sequence
  Description of Artificial Sequence Synthetic oligonucleotide
ctcgagtaaatgaaaatattcaagagctcgagtaaatgaaaatat

SEQ ID NO: 27 - DNA, 21 bp, Artificial Sequence
  Description of Artificial Sequence Synthetic oligonucleotide
agatatcagacctacgggtgc

SEQ ID NO: 28 - DNA, 21 bp, Artificial Sequence
  Description of Artificial Sequence Synthetic oligonucleotide
gagaacggtcctaggcctgaa

SEQ ID NO: 29 - DNA, 21 bp, Artificial Sequence
  Description of Artificial Sequence Synthetic oligonucleotide
tactcgagtaaatgaaaatat

(19) United States
(12) Patent Application Publication
(10) Pub. No.: US 2021/0079402 A1
(43) Pub. Date: Mar. 18, 2021

(54) INHIBITION OF ZSCAN4 FOR INHIBITION OF TUMOR GROWTH

(71) Applicant: University of Maryland, Baltimore, Baltimore, MD (US)

(72) Inventors: Michal Zalzman, Baltimore, MD (US); Scott E. Strome, Reisterstown, MD (US)

(21) Appl. No.: 16/971,242

(22) PCT Filed: Feb. 20, 2019
(86) PCT No.: PCT/US19/18791

§ 371 Date: Aug. 19, 2020

Related U.S. Application Data
(60) Provisional application No. 62/633,060, filed on Feb. 20, 2018.

(51) Int. Cl. C12N 15/113; A61P 35/00; A61K 45/06; A61K 31/475; A61K 31/7088; A61K 31/196; A61K 31/426; C07K 16/40; A61K 39/395

(52) U.S. Cl. CPC C12N 15/1137; A61P 35/00; A61K 45/06; A61K 31/475; A61K 31/7088; C12N 2320/31; A61K 31/426; C07K 16/40; A61K 39/3955; C12N 2310/14; A61K 31/196

(57) ABSTRACT

Embodiments of the disclosure concern methods and compositions for treatment or prevention of cancer, such as through inhibition of the expression and/or activity of a telomere element that promotes telomere extension, such as ZSCAN4. In particular embodiments, inhibition of expression of ZSCAN4 with RNA interference strategies provides inhibition of proliferation of cancer cells that express ZSCAN4. Methods of sensitizing resistant cancers are also provided.

### CROSS-REFERENCE TO RELATED APPLICATIONS

This application claims priority to U.S. Provisional Patent Application Ser. No. 62/633,060, filed Feb. 20, 2018, which is incorporated by reference herein in its entirety.

### SEQUENCE LISTING

The instant application contains a Sequence Listing which has been submitted electronically in ASCII format and is hereby incorporated by reference in its entirety. Said ASCII copy, created on Feb. 19, 2019, is named UOMD_P0025WO_SL.txt and is 7,359 bytes in size.

### TECHNICAL FIELD

Embodiments of the disclosure concern at least the fields of cell biology, molecular biology, and medicine, including at least cancer medicine.

### BACKGROUND OF THE INVENTION

Telomeres are repetitive DNA sequences located at the ends of each chromosome that shorten with every cell division in order to regulate cellular aging, also known as senescence. As such, telomere shortening functions as a biological clock that limits the cell's ability to replicate indefinitely. As long as this clock is intact, replicating cells undergo senescence within approximately 40-50 cell divisions (Hayflick & Moorhead, 1961). Conversely, cancer cells subvert the normal process of aging by activating mechanisms for telomere elongation (Gilley, et al., 2005; Lechel, et al., 2005; Yang, 2008; Bryan, et al., 1997).

Telomere extension has traditionally been attributed to the enzyme telomerase (Greider & Blackburn, 1985; Blackburn, et al., 1989; Shippen-Lentz & Blackburn, 1990), however, additional mechanisms are important. Specifically, studies in cells that lack active telomerase show that telomere regulation is mediated through the long non-coding RNA, TERRA (telomeric repeat-containing RNA) (Azzalin, et al., 2007; Nergadze, et al., 2009; Schoeftner & Blasco, 2008). Indeed, several lines of evidence now demonstrate that short telomeres are transcriptionally active (Yehezkel, et al., 2008; Balk, et al., 2013) and encode for TERRA (Azzalin, et al., 2007; Nergadze, et al., 2009; Schoeftner & Blasco, 2008). TERRA then associates with telomeres to form telomeric DNA-RNA hybrids (Chawla & Azzalin, 2008) whose accumulation leads to telomere lengthening and inhibition of senescence (Balk, et al., 2013; Yu, et al., 2014; Arora, et al., 2014). Moreover, telomeres are preferentially elongated through recombination where DNA-RNA hybrids accumulate; suggesting TERRA marks dysfunctional telomeres for repair (Arora, et al., 2014; Luke, et al., 2008). Importantly, the precise mechanisms controlling TERRA accumulation and association with telomeres are not well understood.

The present disclosure provides solutions to a long-felt need in the art of effective cancer therapy.

### BRIEF SUMMARY

Embodiments of the disclosure concern methods and/or compositions related to cancer treatment or prevention. In particular embodiments, one or more agents are provided to an individual in need thereof for treatment or prevention of cancer of any kind. The individual may be known to have cancer, suspected of having cancer, or at risk for having cancer. The individual may or may not have been diagnosed for the cancer or a specific cancer type and/or grade. In some cases, an individual has a personal and/or family history of cancer. Embodiments of the disclosure include methods of prevention, including for individuals with a personal and/or family history of cancer.

In specific embodiments, the disclosure concerns methods and/or compositions related to the presence of ZSCAN4 in cells of an individual, and in particular embodiments the cells are cancer cells or pre-cancer cells or cancer stem cells. In certain embodiments, the cells previously lacked detectable ZSCAN4 expression and the expression has become detectable, such as when the expression has been altered.

Particular embodiments concern reduction in expression and/or activity of ZSCAN4 in cells of an individual, including, for example, in cells that are undesirable and that have ZSCAN4 expression; examples include cancer cells of any kind. In particular embodiments, the expression and/or activity of ZSCAN4 is reduced by delivering an effective amount of one or more agents to the individual. The one or more agents may be of any kind, although in specific embodiments they reduce expression and/or activity of ZSCAN4. In some embodiments, they reduce expression of ZSCAN4, such as through RNA interference, for example. Such one or more agents may, in certain embodiments, knockdown expression of ZSCAN4, and such knockdown in expression, in certain embodiments, results in a detectable functional or mechanistic event that may or may not be monitored. In specific embodiments, knockdown of ZSCAN4 results in one or more of the following: inhibition of invasion of cancer cells; inhibition of migration of cancer cells; inhibition of tumor formation and growth; inhibition of metastases formation; telomere shortening; onset of senescence; telomere-mediated replicative senescence; reduced cell survival; loss of immortality; reinstatement of cellular aging; loss of regulation of TERRA hybrid accumulation at telomeres; loss of formation of complexes with one or more telomere recombination elements, such as MRE11A, RAD50, or both; loss of promotion of cancer cellular lifespan; and loss of promotion of telomere extension, for example.

Particular embodiments of the disclosure concern modulation of cancer cell growth irrespective of telomerase expression and/or activity, including modulation of cancer cell growth by modulating expression and/or activity of ZSCAN4 in the cancer cell or cancer cell precursor.

Certain embodiments concern methods of imparting no significant increase in tumor grade in existing cancer cells in an individual by reducing, at least in part, expression and/or activity of ZSCAN4 in the cancer cells. Specific embodiments relate to methods of restoring cellular aging in cells (such as cancer cells) that express ZSCAN4 in an individual with cancer by reducing the expression and/or activity of ZSCAN4 in the cells.

In certain embodiments of the disclosure, there are methods of rendering cancer cells sensitive to a cancer therapy (such as chemotherapy, radiation, immunotherapy (including immunomodulatory agents such as checkpoint inhibition, indoleamine 2,3-dioxygenase (IDO), etc.), hormore therapy and/or surgery) by delivering to an individual with the cancer cells an effective amount of one or more agents that reduce the expression and/or activity of ZSCAN4.

Embodiments of the disclosure include methods for growth inhibition of cancer (such as tumors) in mammals by providing to a mammal an effective amount of one or more agents that reduce the expression and/or activity of ZSCAN4 such that the agent provides an effective treatment of malignant disease.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawing, in which:

FIG. 1A-1F—ZSCAN4 is upregulated in cancer. 1A, A representative image of an oral cavity cancer core stained with H&E showing typical tumor nests. 1B, A consecutive slice of the same core, stained with anti-ZSCAN4 (red). 1c, A ×100 magnification showing nuclear ZSCAN4 foci. 1D, A representative image of normal tissue core is ZSCAN4 negative. Nuclei marked by DAPI (blue). 1E, ZSCAN4 is upregulated in a spectrum of cancer cell lines, as shown by qPCR and by 1F, Immunoblot analysis of urea extracted nuclear fraction, whereas normal human tonsil primary cell lines and normal human tonsil tissue controls from five different donors are negative. Error bars indicate S.E.M (p≤0.001).

FIG. 2A-2F—ZSCAN4 binds and extends telomeres. 2A, Immunoblot analyses indicate ZSCAN4 is tightly regulated by Dox and localizes to the nuclear matrix; Controls: LDH (cytosol), Lamin-B (nucleus). 2B, Co-localization of telomeres (red, Cy3-conjugated PNA probe) and ZSCAN4 (green, immunostain) in metaphase spreads. Chromosomes stained with DAPI (blue). 2C, Confocal microscopy analysis reveals that ZSCAN4 preferentially associates with short telomeres (p≤0.01). Study was performed by ImageJ software. Error bars indicate S.E.M. Data based on three independent experiments. 2D, Telomere ChIP assays using anti-ZSCAN4 IgG. Values are relative to 10% DNA input shown as mean±S.E.M from 3 independent experiments; other controls used: negative: Beta-actin, normal IgG; positive: RNA PolII IgG to demonstrate equal loading. Asterisks indicate ** p≤0.001, *** p≤0.0001. 2E, Induction of ZSCAN4 in telomerase positive cells (Tu167), (also see FIG. 14) and 2F, telomerase negative cells (U2OS), leads to telomere extension as measured by qPCR analysis. Results are shown as mean±S.E.M., in at least 6 independent experiments.

FIG. 3A-3D—ZSCAN4 interacts and promotes DNA-RNA hybrids at telomeres. 3A, TERRA ChIP analyses for the indicated subtelomeres, using antibodies against ZSCAN4 and 3B, TERRA DRIP analyses with the DNA-RNA specific S9.6 antibody. RNAse H treatment abolishes the interaction and regulation of DNA-RNA hybrids by ZSCAN4. Values are relative to 10% DNA input shown as mean±S.E.M from 3 independent experiments; other controls used: normal IgG; Beta-actin; positive: RNA PolII IgG to demonstrate equal DNA loading. 3C, Confocal microscope analysis show co-localization of: ZSCAN4 (red) and DNA-RNA hybrids (green; anti-S9.6); and 3D, DNA-RNA hybrids (green; anti-S9.6) with the telomere marker TRF1 (red). Analyses by imageJ software confirms 71.4±6.1% (mean±S.E.M) of the DNA-RNA hybrids following ZSCAN4 induction, localize at telomeres (p≤0.005). Nuclei marked by DAPI (blue). Size bar=10 μm. *** Asterisks indicate p≤0.0001, ** p≤0.001.

FIGS. 4A-4F—ZSCAN4 forms a complex with recombination mediators, and its depletion sequesters them of the telomeres. 4A, The interaction of ZSCAN4 with the recombination mediators RAD50 and MRE11 is demonstrated by co-immunoprecipitation (Co-IP) of nuclear protein lysates with anti-ZSCAN4, followed by immunoblot of MRE11 or RAD50. 4B, Reverse Co-IP with anti-MRE11 or 4C, anti-RAD50 antibodies confirms ZSCAN4 forms a complex with these recombination enzymes. IgG was used as control. 4D, Immunoblot analyses show ZSCAN4 knockdown (KD) leads to significantly reduced levels of MRE11 and RAD50. 4E-4F, A significant decrease in telomere associated RAD50 and MRE11 is demonstrated by co-localization analyses of telomeres FISH (red, Cy3-conjugated PNA probe) and anti-RAD50 (green, immunostain) or 4F, anti-MRE11 in metaphase spreads. Chromosomes stained with DAPI (blue). Asterisks indicate ** p≤0.01, # indicate a marginal effect (p≤0.054).

FIG. 5A-5B—Increase in telomere recombination events (arrows) shown by CO-FISH (and FIG. 15); The leading strand telomeres were revealed by 3′ Alexa546-DNA probe (red) and the lagging strand telomeres by a 5′ Alexa488-PNA probe (green); chromosomes stained with DAPI (blue). Left, non-induced cells (Dox−; 5A). Right, ZSCAN4-induced cells (Dox+; 5B). Table: A summary of total telomere recombination (T-Rec). Recombination events were counted n≥1139; n=number of chromosomes per sample from three independent experiments.

FIGS. 6A-6G. Knockdown of ZSCAN4 ultimately leads to population growth arrest. (6A) Illustration of ZSCAN4 knockdown vector used to transfect Tu167 and 012SCC cancer cell lines. (6B) Confirmation of ZSCAN4 knockdown by all four ZSCAN4 shRNA sequences (shRNA1-shRNA4) in Tu167 cells as shown by real-time qRT-PCR analysis. No significant (n.s) effect in ZSCAN4 expression was detected in NTC-shRNA control compared to isogenic cells transfected with Empty vector (same vector with no shRNA). *** Asterisks indicate p≤0.001. (6C) Representative images of ZSCAN4 immunostaining (green) in knockdown and isogenic controls. Panel shows nuclei of cells transfected with shRNA1-4. As controls we used cells transfected with Empty vector and cells expressing non-targeting control shRNA (NTC-shRNA). Scale bar=10 μm. Nuclei are stained by DAPI (blue). Arrow mark ZSCAN4 foci. (6D) Population doubling assays show ZSCAN4 depletion leads to reduction of proliferation in Tu167 cells and (6E) 012SCC cells, and with passage, to loss of self-renewal potential. (6F, 6G) Increase in in G2/M arrested cells observed at late passages in ZSCAN4 knockdown (Tu167 and 012SCC respectively) cells compared to NTC-shRNA isogenic controls. Cell cycle analysis by flow cytometry (n=8 per sample) after 15 PDs for Tu167 and 10 PDs for 012SCCs. Percentage of cells in each phase of cell cycle were analyzed by FlowJo software. Asterisks *** indicate p<0.001, **** p<0.0001.

FIGS. 7A-7F. Knockdown of ZSCAN4 leads to telomere-mediated cellular senescence. (7A) ZSCAN4 depletion in Tu167 and (7B) 012SCC cells shows reduced telomere length with population doublings (PD) due to ZSCAN4 knockdown via telomere qPCR analysis. Relative telomere length ratios where compared to a single copy gene (T/S). All data shown as mean±S.E.M. observed in triplicate in at least three independent experiments. Asterisks indicate: *p≤0.05, **p≤0.01, ***p≤0.001. (7C) Representative images in ×40 magnification in Ph II showing histochemical analyses by Sudan black B (dark blue) in TU167 and (7D) 012SCC, and validated by histochemical analysis with the senescence associated beta-galactosidase activity assay (blue stain) in (7E) TU167 and (7F) 012SCC show ZSCAN4 deficient cultures (ZSCAN4 knockdown) accumulate senescent cells in late passages while early passages and isogenic NTC-shRNA cells remain negative for both. Size bar=20 μm.

FIGS. 8A-8G. ZSCAN4 depletion severely affects tumor growth. (8A) Schematic illustration of mouse xenograft model. NGS mice were injected subcutaneously with Tu167 ZSCAN4 knockdown cells (n=10), or NTC-shRNA cells as controls (n=10) and allowed to form xenograft tumors. (8B) Tumor volume at indicated time. Error bars denote S.E.M., (p≤0.001) starting from week 3. (8C) Kaplan-Meier survival curve of mice inoculated (p≤0.001); results are shown from day of cell injection to day of euthanasia. (8D) Representative images of NTC-shRNA control and ZSCAN4 depleted tumors harvested 36 days following inoculation. (8E) Pictures show individual tumors, still attached to the mouse skin. Control cells (NTC-shRNA and Empty vector) gave a tumor within 5 weeks. Tumors of ZSCAN4 knockdown cells remained attenuated and were harvested at two time points: 5 and 15 weeks after inoculation. Arrows mark indicated tumor size in mm. (8F) Representative images (n=6 per group), showing increase in senescence in the tumor nests of ZSCAN4 depleted cells compared to controls as shown by GLB1 immunostaining (red) with corresponding consecutive slide stained by H&E. Nuclei are stained with DAPI (blue). Size bar=200 μm. Results are validated by (8G) Sudan black B (n=6 per group) from a separate tumor slice showing dark blue stain (in Phase I), only in tumor nests of ZSCAN4 depleted cells, whereas NTC-shRNA control remain unstained.

FIGS. 9A-9D. ZSCAN4 associates with and form foci on the telomere region (9A) tet-ZSCAN4 expression vector. (9B) Image of tet-ZSCAN4 (Tu167) cells in phase contrast showing GFP (green). (9C) Immunoblot analyses indicate ZSCAN4 is tightly regulated by Dox and localizes to the nuclear matrix; Controls: LDH (cytosol), Lamin-B (nucleus). (9D) Histograms obtained from cell cycle analysis by flow cytometry show no significant difference in cell cycle following ZSCAN4 induction by Dox. Percentage of cells in each phase of cell cycle were analyzed by FlowJo software; n=10 in 3 independent experiments. Error bars indicate S.E.M. ** Asterisks indicate p≤0.01.

FIGS. 10A-10G. ZSCAN4 regulates telomere length. (10A) Induction of ZSCAN4 for 72 hours leads to a 1.8 fold telomere extension in telomerase positive Tu167 cells as demonstrated via telomere length qPCR analysis. Confirmation of telomere extension by Q-FISH analysis shows a distribution diagram of relative telomere length, of: (10B) non induced tet-ZSCAN4 cells (Dox−) and (10C) Dox treated tet-ZSCAN4 (Dox+) induced cells as analyzed by TFL-Telo software (results of pooled nuclei, totaling >2500 telomeres). TFU=telomere fluorescent unit. (10D, 10E) Representative images of corresponding metaphase spreads stained for telomere FISH. Telomeres were revealed by Alexa546-DNA probe (red). Chromosomes stained with DAPI (blue). Induction of ZSCAN4 by Dox in: (10F) breast cancer (SKBR3) and (10G) colorectal cancer (SW480) cell lines, leads to telomere extension as measured by telomere qPCR analysis. Results are shown as mean±SEM, demonstrated repeatedly in at least 3 independent experiments. Data were analyzed by one-way ANOVA. **Asterisks indicate p≤0.01, * indicate p≤0.05.

FIGS. 11A-11D. ZSCAN4 extends telomeres in telomerase negative cells (11A) Induction of ZSCAN4 in telomerase negative cells (U2OS), leads to telomere extension as measured by qPCR analysis. Results are shown as mean±S.E.M., in at least 6 independent experiments. (11B) Q-FISH analyses validates ZSCAN4 facilitates extension of telomeres in the absence of telomerase. A distribution diagram of relative telomere length of uninduced tet-ZSCAN4 U2OS cells (Dox−) and (11C) Dox treated tet-ZSCAN4 (Dox+) induced cells as analyzed by TFL-Telo software (results of pooled nuclei, totaling >2000 telomeres). TFU=telomere fluorescent unit. Data was reproduced in three independent experiments. (11D) Terminal restriction fragment length measurement by southern blot analysis show telomere extension following 3 days of Zscan4 induction (Dox+). DNA bands were detected by hybridization with a 3′ biotin-labeled telomere probe. Isogenic wild type (WT) U2OS cells in the presence (Dox+) of absence of Dox (Dox−) and uninduced U2OS tet-ZSCAN4 (Dox−) were used as controls. Data was reproduced in four independent experiments.

FIGS. 12A-12E. ZSCAN4 does not affect telomerase activity. Validation of telomerase status across multiple cell lines tested: qPCR data demonstrates expression levels of (12A) hTERT and (12B) hTERC in telomerase positive (Tu167, 012SCC, SKBr3, SW480) and telomerase negative (U2OS) cells. Hela cells were used as telomerase positive control. (12C) Telomerase activity (TRAPeze RT) qPCR assay confirm telomerase status in our cell lines. (12D) ZSCAN4 induction does not lead to increase in telomerase activity, and a mild reduction is observed as shown by Telomerase TRAPeze RT real time qPCR. Controls: isogenic wild type (WT) Tu167 cells in untreated and Dox treatment conditions. No significant effects on telomerase activity were detected. (12E) ZSCAN4 knockdown does not lead to a significant change in telomerase activity throughout populations doublings (PD) in TU167 HNSCC cell line. Telomerase TRAPeze RT qPCR assay demonstrates telomerase activity remains high up to 20PD (compared to Hela cells used as calibrating positive control). Additional controls used: isogenic NTC-shRNA cells in equivalent passages, isogenic WT cell extracts. No significant effects on telomerase activity were indicated. For all TRAPeze RT assays shown here (12D-12E) additional technical controls uses (not shown): telomerase positive HeLa cells. Negative controls used: heat inactivated extracts per each sample. Error bars indicate S.E.M.

FIGS. 13A-13B—13a, ZSCAN4 effect on TERRA expression. 13A, Real time RT-qPCR analysis using primers for ZSCAN4 and for TERRA expressed from three different chromosomes show Dox induction of ZSCAN4 does not significantly affect TERRA expression. ***Asterisks indicate p≤0.0001. Error bars indicate S.E.M. 13B, ZSCAN4 promotes RNA-DNA hybrids on telomeres in telomerase negative cells (U2OS). TERRA DRIP analysis in ZSCAN4 inducible (Dox) ALT cells (U2OS) using RNA-DNA hybrids specific S9.6 antibody. Values are represented as percentage input of TERRA DNA recovered; data shown as means±SEM in 3 biological replicates. Data was reproduced in at least 3 independent experiments. ***Asterisks indicate p≤0.0001. **Asterisks indicate p≤0.001.

FIG. 14: ZSCAN4 promote telomere recombination. Whole metaphase image of CO-FISH following ZSCAN4 induction. Telomere recombination events (arrows) are demonstrated; The leading strand telomeres were revealed by 3′ Alexa546-DNA probe (red) and the lagging strand telomeres were revealed by a 5′Alexa488-PNA probe (green). Chromosomes stained with DAPI (blue).

FIG. 15A-15C—ZSCAN4 depletion leads to telomere mediated tissue culture crisis. Representative Phase I images+RFP (red) of non-targeting control shRNA and ZSCAN4 depleted 012SCC cells at 15A, Early passages (P+3, 7 population doublings (PD)) after ZSCAN4 knockdown and 15B, Late passage (P+10, approximately 15 PD); right: tissue culture crisis of ZSCAN4 knockdown cells showing detached dead cells floating in the culture medium 15C, Representative images of metaphase spreads of cells one passage prior to culture crisis (approximately 15 PD) stained for Telomere FISH by Cy3-conjugated telomere DNA probe (red); chromosomes are marked by DAPI (blue): ZSCAN4 knockdown cells display significantly shorter telomeres (white arrows); (right) isogenic NTC-shRNA control cells (ZSCAN4 positive). Size bar=10 μm.

FIG. 16—ZSCAN4 deficiency severely affects HNSCC tumor growth in vivo. Pictures show individual tumors, still attached to the mouse skin. Control cells (NTC-shRNA and Empty vector) gave a large tumor within 36 days. Tumors of ZSCAN4 knockdown cells remained attenuated and were harvested at two time points: 5 and 15 weeks after inoculation. Arrows mark indicated tumor size in mm.

FIGS. 17A-17 F. Human ZSCAN4 protein half-life. 17A. Immunoblot analysis of ZSCAN4 in wild type (WT) Tu167 cells after treatment with cycloheximide (CHX) results in decreased ZSCAN4 protein. 17B. Protein half-life analysis indicates that endogenous ZSCAN4 protein half-life is 8.3 h. 17C. Immunoblot in tet-ZSCAN4 cell lines show addition of doxycycline (Dox+) to medium for 0-48 hours results in ZSCAN4-FLAG induction in as early as 12 h. 17D. Cells fractionation to cytoplasmic (C) and nuclear (N) proteins, show the endogenous ZSCAN4 (anti-ZSCAN4) and FLAG-tagged ZSCAN4 (anti-FLAG), are localized to the nucleus. 17E. Immunoblot in isogenic (Dox+) tet-ZSCAN4 cells after treatment with CHX and 17F. Protein half-life analyses indicate that the ectopic ZSCAN4 half-life is 8.0 h. Images and results represent data of at least three independent experiments.

FIGS. 18A-18 E. ZSCAN4 degradation is proteasome dependent 18A. Immunoblot analyses indicate that inhibition of autophagy with Bafilomycin A1 and Chloroquine results in accumulation of autophagy targets p62 and LC-3. However, ZSCAN4 was not affected. 18B. Induction of ZSCAN4 by Dox followed by treatment with the proteasomal inhibitor MG132 demonstrates ZSCAN4 accumulates in the cells. 18C-18D. Kinetics experiments show that accumulation of ZSCAN4 following by MG132 treatment is time and dose dependent. E Similar studies performed in WT cells show the accumulation of ZSCAN4 following MG132 treatment.

FIGS. 19A-19E. ZSCAN4 is Lysine 48 (K48Ub) polyubiquitinated 19A. Endogenous ZSCAN4 immunoprecipitation (IP) in treated (+MG132) or untreated (−MG132) WT cells followed by ZSCAN4 immunoblot. 19B. Denaturing conditions where used followed by ZSCAN4-IP and immunoblot with anti-Lysine 48 (K48) ubiquitin (anti-K48Ub). MG132 treated cells indicate that ZSCAN4 is polyubiquitinated. Untreated cells were used as controls. 19C. Co-immunostaining and confocal microscopy analyses in WT Tu167 cells treated with MG132 (+MG132) or untreated (−MG132) using anti-ZSCAN4 (green) and anti-K48 ubiquitin (red) indicates the ZSCAN4 overlaps with K48Ub. 19D. Colocalization analyses with ImageJ show an increase in the number of ZSCAN4 foci colocalized with K48Ub and 19E. the percent (%) of K48Ub colocalized ZSCAN4 foci (n=6 per group and average of >300 foci per group). Asterisks indicate * p<0.05, ** p<0.01.

FIGS. 20A-20BD. ZSCAN4 depletion leads to loss of MRE11 and RAD50 at the telomeres. (20A) Co-localization of telomeres (red, Cy3-conjugated PNA probe) and MRE11 (green, immunostain) in metaphase spreads of ZSCAN4 knockdown and NTC shRNA (in tu167 cell line). Chromosomes stained with DAPI (blue). (20B) Co-localization of telomeres (red, Cy3-conjugated PNA probe) and RAD50 (green, immunostain) in metaphase spreads. Chromosomes stained with DAPI (blue). (20C) Confocal microscopy analysis reveals a significant reduction in MRE11 foci at telomeres but not genomic regions. Error bars indicate S.E.M. Data based on three independent experiments. (20D) Confocal microscopy analysis reveals a significant reduction in RAD50 foci at telomeres but not genomic regions. Error bars indicate S.E.M. Data based on three biological replicates (n=6 images per group). ** Asterisks denote p<0.01. # denotes a marginal effect p=0.056.

FIGS. 21A-21D. ZSCAN4 requires MRE11 to extend telomeres. (21A) Q-FISH analyses validates ZSCAN4 extends telomeres. (21B) ZSCAN4 mediated telomere extension is reduced after MRE11 inhibition with Mirin. A distribution diagram of relative telomere length of non-induced tet-ZSCAN4 U2OS cells (Dox−) and Dox treated tet-ZSCAN4 U2OS (Dox+) induced cells as analyzed by TFL-Telo software (results of pooled nuclei, totaling >2000 telomeres). TFU=telomere fluorescent unit. (21C) Demonstrates mean values of histogram as well as average number of telomere signals per cell analyzed. (21D) Induction of ZSCAN4 in tet-ZSCAN4 U2OS cells after Mirin treatment, leads to telomere extension only in the mock (DMSO) treated samples, but not in Mirin treated cells. Results are shown as mean±S.E.M. *** Asterisks denotes p<0.005.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the invention may “consist essentially of” or “consist of” one or more sequences of the invention, for example. Some embodiments of the invention may consist of or consist essentially of one or more elements, method steps, and/or methods of the invention. It is contemplated that any method or composition described herein can be implemented with respect to any other method or composition described herein. Embodiments discussed in the context of methods and/or compositions of the invention may be employed with respect to any other method or composition described herein. Thus, an embodiment pertaining to one method or composition may be applied to other methods and compositions of the invention as well.

The present disclosure concerns the activity of the human ZSCAN4 and its role in the maintenance of cancer cell immortality. ZSCAN4 is reactivated in human cancers and cancer cell lines and plays a role in telomere extension by promoting TERRA RNA-DNA hybrid accumulation as well as recombination events at telomeres. In specific embodiments, the activity of ZSCAN4 in the maintenance of tumor growth and survival is utilized as a target for cancer therapeutics. In addition, ZSCAN4 protein turnover in humans is demonstrated. The half-life of the stem cell factor and telomere regulator ZSCAN4 is demonstrated as 8.0 hours.

I. Methods of Treatment or Prevention

In one embodiment, one or more ZSCAN4-inhibiting agents are used for the prevention, treatment or amelioration of a cancerous disease, such as a tumorous disease. The ZSCAN4-inhibiting agent may be formulated in a pharmaceutical composition. In particular embodiments, the pharmaceutical composition contemplated herein may be particularly useful in preventing, ameliorating and/or treating cancers (or ameliorating one or more symptoms thereof). In specific embodiments, cancer cells being treated with pharmaceutical compositions are effectively treated because ZSCAN4-inhibiting agents of the pharmaceutical compositions are selective for ZSCAN4-expressing cells. In particular embodiments, the cancer is in the form of a solid tumor, although the cancer may be non-solid in nature.

As used herein “treatment” or “treating,” includes any beneficial or desirable effect on the symptoms or pathology of a disease or pathological condition, and may include even minimal reductions in one or more measurable markers of the disease or condition being treated, e.g., cancer. Treatment can involve optionally either the reduction or amelioration of symptoms of the disease or condition, or the delaying of the progression of the disease or condition. “Treatment” does not necessarily indicate complete eradication or cure of the disease or condition, or associated symptoms thereof.

As used herein, “prevent,” and similar words such as “prevented,” “preventing” etc., indicate an approach for preventing, inhibiting, or reducing the likelihood of the occurrence or recurrence of, a disease or condition, e.g., cancer. It also refers to delaying the onset or recurrence of a disease or condition or delaying the occurrence or recurrence of the symptoms of a disease or condition. As used herein, “prevention” and similar words also includes reducing the intensity, effect, symptoms and/or burden of a disease or condition prior to onset or recurrence of the disease or condition.

Components upstream of ZSCAN in a pathway include at least Tbx3; Dux4; PI3-Kinase; LSD1; CtBP2; and/or TERRA. In some embodiments, one or more of these gene products may be modulated in level and/or activity to affect ZSCAN regulation.

Components downstream of ZSCAN in a pathway include at least Uhrf1; Dnmt1; histone deacetylases; histone acetylases; and human counterparts of Tmem92, Tcstv3, Gm428, Thoc4. One or more of such gene products may be modulated in level and/or activity in certain methods of the disclosure.

In particular embodiments, the administration of the ZSCAN4-inhibiting agent composition(s) of the disclosure is useful for all stages and types of cancer, including for minimal residual disease, early cancer, advanced cancer, and/or metastatic cancer and/or refractory cancer, for example. The treatments may inhibit metastasis of cancer or delay the onset or severity of metastatic cancer. The disclosure further encompasses co-administration protocols with other compounds that are effective against cancer. The clinical regimen for co-administration of the inventive agent(s) may encompass co-administration at the same time, before, or after the administration of the other component. Particular combination therapies include chemotherapy, radiation, surgery, hormone therapy, immunotherapy, or a combination thereof.

Embodiments of the disclosure concern the treatment or prevention of cancer in a mammal by providing to the mammal an effective amount of at least one ZSCAN4-inhibiting agent. In particular embodiments the cancer includes cells that express ZSCAN4. The ZSCAN4-inhibiting agent(s) may be of any kind, but in specific embodiments, the ZSCAN4-inhibiting agent is a nucleic acid (RNA, DNA, or both); a protein or polypeptide; a small molecule; an agent that will prevent its interaction with DNA-RNA hybrids, the telomeres or G-quadruplexes; or a combination thereof. The mechanism of action of the agent(s) may be of any kind, including by directly or indirectly inhibiting expression of ZSCAN4 and/or by directly or indirectly inhibiting activity of ZSCAN4. In embodiments wherein expression of ZSCAN4 is inhibited, the inhibition may be from complementary binding of a nucleic acid with at least part of the ZSCAN4 transcript. Regardless of the type of ZSCAN4-inhibiting agent, the inhibition of expression or activity may be through the Zinc finger domain and/or the SCAN domain, in certain cases. The part of the ZSCAN4 transcript to which it binds may be of any region, including exon, intron, untranslated region, or a combination thereof. In embodiments wherein inhibition of expression of ZSCAN4 occurs indirectly, the inhibition may be, for example, through inhibition of the binding of one or more transcription factors to a regulatory region that modulates expression of ZSCAN4. Inhibition of the ZSCAN4 complex formation or inhibition of its binding to DNA-RNA hybrids or G-quadruplexes. The regulatory region may be outside (upstream or downstream) of the ZSCAN4 gene or it may be within the coding region.

In specific embodiments, the ZSCAN4-inhibiting agent may act through inhibition of formation of DNA-RNA hybrids at the cancer cell telomere(s). Telomeric repeat-containing RNA (TERRA) is a long non-coding RNA that forms part of telomeric heterochromatin and is involved in maintaining the telomeric structure, and in specific embodiments the ZSCAN4-inhibiting agent interferes with activity involving ZSCAN4 and TERRA. In specific embodiments, the ZSCAN4-inhibiting agent interferes with transcription of TERRAs from the telomeric end of DNA in cancer cells. In particular embodiments, the ZSCAN4-inhibiting agent interferes with binding of one or more telomeric-binding proteins (including single-strand DNA telomere binding proteins and double-strand DNA telomere binding proteins) to a cancer cell telomere. Examples of telomeric-binding proteins include TRF1, TRF2, Pot1, hTERT, RAP1, and in specific embodiments the ZSCAN4-inhibiting agent interferes with the binding of one or more of them to the telomere. In certain embodiments, the ZSCAN4-inhibiting agent interferes with activity of one or more recombination enzymes and ZSCAN4-interacting factors at the telomere, such as MRE11A, RAD50, DMC1, SPO11, the HNRNP family factors or the interaction of ZSCAN4 with DNA-RNA hybrids, and/or DNA G-quadruplexes, for example.

Particular embodiments of the disclosure include the inhibition of ZSCAN4 expression and/or activity by providing an effective amount of a polypeptide or peptide to cancer cells of an individual such that the polypeptide or peptide inhibits a particular region of ZSCAN4, an particular region of an agonist of ZSCAN4, and/or inhibits expression of ZSCAN4, such as through inhibition of one or more transcription factors. The activity of the polypeptide or peptide may include inhibition of binding of ZSCAN4 to another element or complex of elements, such as the telomere or a telomere-binding component(s) thereof.

In specific embodiments, one or more small molecules are utilized for inhibition of ZSCAN4 expression and/or activity in cancer cells of an individual. The small molecule may be of any kind, but in specific embodiments the small molecule has an inhibitory effect of the ability of ZSCAN4 to bind DNA-RNA hybrids such as DNA-RNA binding ligands, or factors and small molecules that will interfere with the ability of ZSCAN4 complex to bind to G-Quadruplexes, such as G4 ligands and targeting molecules, for example.

In specific embodiments, knockdown of ZSCAN4 by a ZSCAN4-inhibiting agent results in one or more of the following: telomere shortening; onset of senescence; telomere-mediated replicative senescence; reduced cell survival; loss of immortality; reinstatement of cellular aging; loss of regulation of TERRA hybrid accumulation at telomeres; loss of formation of complexes with one or more telomere recombination elements, or the HNRNP family factors such as MRE11A, RAD50, DMC1, SPO11, the HNRNP family factors or the interaction of ZSCAN4 with DNA-RNA hybrids, and DNA G-quadruplexes, or more than one of those; loss of promotion of cancer cellular lifespan; population growth arrest; tumor growth arrest or reduction; loss of promotion of telomere extension; and/or changes in heterochromatin into transcribed chromatin markers, such as H3K27ac, H3K9ac, H3K8ac, H4K8ac, for example. The skilled artisan recognizes from routine methods in the art how to test for the function of such parameters. For example, accumulation of autolysosomes and/or autophagosomes measures senescence and/or a SA-Gal activity assay, for example, may be utilized to measure senescence.

Any individual may be treated with methods and/or compositions of the disclosure. The individual may be of any gender and of any age. The individual may or may not have been diagnosed for the cancer. The individual may be known to have cancer, suspected of having cancer, or at risk for having cancer. The individual may be at risk for having cancer by having a personal or family history, by being overweight or a tobacco user, by exposure to chemicals and/or sun and/or radiation, by having a genetic predisposition, by aging, by exposure to some viruses and bacteria or certain hormones, by having a poor diet or lack of sufficient physical activity, by having one or more genetic markers associated with cancer, by having had exposure to one or more carcinogens and/or ultraviolet rays, or a combination thereof, for example.

In certain cases, the individual has malignant or benign tumors. These could be malignancies and cancers related at least to brain, stomach, head and neck, skin, thyroid, lung, gastrointestinal tract, genitourinary tract, pancreatic, breast, ovarian, testicular, liver, urethra, bone, neuronal, gynecological cancers, hematologic, adrenal gland cancers, epithelial, prostate, non-small cell lung, cervical, endometrial, or colon cancer. The cancer may be primary, metastatic, refractory, recurrent, relapsed, and so forth.

In certain embodiments, the onset of cancer is prevented in an individual upon exposure to one or more ZSCAN4-inhibiting agents. The exposure may occur in an individual upon routine medical practices or because the individual has a risk of having cancer, for example greater than the general population, or is of an advanced age, for example.

The exposure of the individual to the one or more ZSCAN4-inhibiting agents (whether for treatment or prevention) may occur once or may occur multiple times, and in cases where there are more than one administrations, the duration between successive administrations may be of any suitable time. In specific embodiments, the time span between administrations to prevent and/or treat the cancer is on the order of minutes, hours (such as spanning 1, 2, 3, 4, 5, 6, 7, 8, 9, 10, 11, 12, 13, 14, 15, 16, 17, 18, 19, 20, 21, 22, 23, or 24 hours), days (such as spanning 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or more), weeks (such as spanning 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, or more), months (such as spanning 1, 2, 3, 4, 5, 6, 7, 8, 9, 10, 11, 12, or more), or years (such as spanning 1, 2, 3, 4, 5, 6, 7, 8, 9, 10 or more).

The administration of the one or more ZSCAN4-inhibiting agents may reduce expression of endogenous ZSCAN4 by any detectable level as measured by any standard means, including by at least 2-fold, 5-fold, 10-fold, 25-fold, 50-fold, 100-fold, 250-fold, 500-fold, 750-fold, 1000-fold, and so on in expression level.

II. Compositions

Compositions related to the present disclosure include one or more ZSCAN4-inhibiting agents to reduce the expression and/or activity of ZSCAN4 in cells that express ZSCAN4. The ZSCAN4-inhibiting agent may be a single compound or a plurality of similar, identical, or non-identical compounds. The ZSCAN4-inhibiting agent may be one or more of a nucleic acid of any length, polypeptide, peptide, small molecule, nanoparticle, or a combination thereof.

In embodiments wherein the ZSCAN4-inhibiting agent comprises a nucleic acid, the nucleic acid may be a polynucleotide or an oligonucleotide, or a mixture thereof. The nucleic acid may comprise DNA, RNA, or DNA-RNA hybrid compositions or DNA-RNA mixtures. In specific embodiments, when the ZSCAN4-inhibiting agent comprises a nucleic acid, the nucleotide may be RNA, DNA, viral agent, combined with or in a nanoparticle, etc. When the ZSCAN4-inhibiting agent is a nucleic acid, the nucleic acid may be an RNA interference molecule that acts by binding to a corresponding complementary region on the ZSCAN4 mRNA. The region to which the RNA interference molecule may bind may be any region of the ZSCAN4 mRNA, including one or more of an untranslated region, regulatory region downstream or upstream to the gene, promoter, exon, or intron. The complementarity may be complete, or there may be one or more mismatches (such as 1, 2, 3, 4, 5, 6, 7, 8, 9, 10, or more mismatches, for example) between the RNA interference molecule and its ZSCAN4 mRNA target region, so long as the binding is robust enough to allow the molecules to maintain hybridization. When there is less than 100% complementarity between the RNA interference molecule and its ZSCAN4 mRNA target region, the percentage complementarity may be at least 99, 98, 97, 96, 95, 94, 93, 92, 91, 90, 87, 85, 83, 80, 77, 75, or 70% complementarity. When the ZSCAN4-inhibiting agent is an RNA interference molecule, the RNA interference molecule may be a shRNA, siRNA, miRNA, and so forth. In specific embodiments, the ZSCAN4-inhibiting agent is a shRNA nucleic acid of no more than or no less than 100, 95, 90, 85, 80, 75, 70, 65, 60, 55, or 50 nucleotides in length and, in some cases comprise 1, 2, 3, 4, or 5 mismtaches with its ZSCAN4 mRNA target region. In certain embodiments, the ZSCAN4-inhibiting agent is a siRNA molecule that is no more than or no less than 18, 19, 20, 21, 22, 23, 24, 25, 26, 27, 28, 29, or 30 nucleotides in length and, in some cases comprise 1, 2, 3, 4, or 5 mismtaches with its ZSCAN4 mRNA target region. The ZSCAN4-inhibiting agent may be any of the following sequences or functional variants thereof having the ability to knock down or knock out ZSCAN4 expression or activity:

An example of a ZSCAN4 siRNA is 5′ CCCUAAUCAUCAUCCAGGAtt 3′ (SEQ ID NO:20)

Examples of ZSCAN4 shRNAs are as follows:

ZSCAN4 shRNA1
(SEQ ID NO: 21)
GAGAACGGTCCTAGGCCTGTCAAGAGGAGAACGGTCCTAGGCCTG
ZSCAN4 shRNA2
(SEQ ID NO: 25)
GATATCAGACCTACGGGTGTCAAGAGGATATCAGACCTACGGGTG
ZSCAN4 shRNA3
(SEQ ID NO: 26)
CTCGAGTAAATGAAAATATTCAAGAGCTCGAGTAAATGAAAATAT

Examples of locked nucleic acid siRNAs are as follows:

LNA siRNA1
(SEQ ID NO: 27)
agatatcagacctacgggtgc
LNA siRNA2
(SEQ ID NO: 28)
gagaacggtcctaggcctgaa
LNA siRNA3
(SEQ ID NO: 29)
tactcgagtaaatgaaaatat

Any functional variant of these specific sequences may be utilized including a variant having a sequence that is 1, 2, 3, 4, or 5 nucleotides that are different with respect to these sequences. Any functional variant is at least 90%, 91%, 92%, 93%, 94%, 95%, 96%, 97%, 98%, or 99% identical in sequence to SEQ ID NO:20, SEQ ID NO:21, SEQ ID NO:25, SEQ ID NO:26, SEQ ID NO:27, SEQ ID NO:28, or SEQ ID NO:29, respectively.

The ZSCAN4-inhibiting agent(s) may be formulated in a composition suitable for use in a mammal, including at least a human, dog, cat, horse, cow, goat, sheep, pig, and so forth.

III. Combination Therapy

In order to increase the effectiveness of one or more ZSCAN4-inhibiting agent(s), it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the agent(s) and a second therapy, whether or not at the same time. This may be achieved by contacting the cell with a single composition or pharmacological formulation that includes both agents, or by contacting the cell with two distinct compositions or formulations, including at the same time, wherein one composition includes the ZSCAN4-inhibiting agent(s) and the other includes the second agent(s).

In the context of the present disclosure, it is contemplated that one or more ZSCAN4-inhibiting agent(s) could be used in conjunction with chemotherapeutic, radiotherapeutic, immunotherapeutic, surgery intervention, etc., in addition to other pro-apoptotic or cell cycle regulating agents.

In some embodiments, the one or more ZSCAN4-inhibiting agent(s) may precede or follow the other agent treatment by intervals ranging from minutes to weeks. In embodiments where the other agent and ZSCAN4-inhibiting agent(s) are applied separately to the cell, one would generally ensure that a significant period of time did not expire between the time of each delivery, such that the second agent and ZSCAN4-inhibiting agent(s) would still be able to exert an advantageously combined effect on the cell. In such instances, it is contemplated that one may contact the cell with both modalities within about 12-24 hours of each other and, more preferably, within about 6-12 hours of each other. In some situations, it may be desirable to extend the time period for treatment significantly, however, where several days (2, 3, 4, 5, 6 or 7) to several weeks (1, 2, 3, 4, 5, 6, 7 or 8) lapse between the respective administrations.

Administration of the therapeutic ZSCAN4-inhibiting agent(s) of the present disclosure to a patient may follow general protocols for the administration of chemotherapeutics, taking into account the toxicity, if any, of the agent(s). It is expected that the treatment cycles would be repeated as necessary. It also is contemplated that various standard therapies, as well as surgical intervention, may be applied in combination with the described hyperproliferative cell therapy.

In some embodiments of the disclosure, one or more ZSCAN4-inhibiting agents are utilized in conjunction with one or more other therapies for treatment or prevention of cancer. Although the additional therapy or therapies may be of any kind, in specific embodiments it comprises surgery, radiation, chemotherapy, hormone therapy, immunotherapy, gene therapy, and so forth.

Any one or more ZSCAN4-inhibiting agents may be combined with any one or more additional cancer therapies and/or other agents. The additional therapy may be one or more nucleic acids, peptides, proteins, small molecules, or combinations thereof, for example. In specific embodiments, the additional cancer therapy comprises one or more G-quadruplex ligands, one or more G-quadruplex binders, one or more telomerase inhibitors, one or more inhibitors of MRE11, one or more inhibitors of RAD50, or a combination thereof. The unique structure of G-quadruplex DNA allows specific recognition by small ligands. These ligands can intercalate at GpG steps, be stacked on the terminal G-tetrads, and/or bind in the grooves. Examples of quadruplex ligands include N,N′-bis[2-1(-piperidino)-ethyl]-3,4,9,10-perylene-tetracarboxylic diimide (PIPER); this ligand does not alter the structure of the core quadruplexes or intercalate within the G-quadruplex itself. It is stacked on the surface of the terminal quartet. (Jean-Louis Mergny, “G-quadruplex DNA: A target for drug design. Nature Medicine 4, 1366-1367 (1998)”). Other molecules with an extended aromatic ring that are capable of intercalating with G-tetrads DNA were shown to stabilize G-quadruplexes and inhibit telomerase and can act as ZSCAN4 inhibitors. Examples include dibenzophenanthroline derivatives, 3,6,9-tri-substituted acridine inhibitors, Tetra-(N-methyl-4-pyridyl) and tetra-(N-methyl-2-pyridyl)-porphyrins, 2,6-disubstituted anthraquinones and 2,7-disubstituted fluorenones. Examples of quadruplex binding proteins includes BRCA1, breast cancer type 1 susceptibility protein; hnRNP, heterogeneous nuclear ribonucleoprotein family; POT1, protection of telomeres 1; RPA, replication protein A; TEBP, Telomere End Binding Protein; TLS/FUS, translocated in liposarcoma/fused in sarcoma; Topo I, Topoisomerase I; TRF2, telomere repeat binding factor 2; and UP1, unwinding protein 1.

Specific examples of telomerase inhibitors that may be combined with one or more ZSCAN4-inhibiting agents include at least telomerase inhibitor IX, Imetelstat (GRN163L), BIBR 1532, or a combination thereof. In addition, or as an alternative, one or more ZSCAN4-inhibiting agents are provided to an individual in need thereof with an effective amount of one or more inhibitors of MRE11, RAD50, or both. Examples of MRE11 inhibitors includes Mirin (Z-5-(4-hydroxyphenylidene)-2-imino-1,3-thiazolidin-4-one); Z-5-(4-hydroxybenzylidene)-2-thioxo-1,3-imidazolidin-4-one; and/or 6-phenyl-2-thioxo-2,3-dihydro-4(1H)-pyrimidinone (5122443). Specific examples of MRE11 and RAD50 are described in WO 2010075372, which is incorporated by reference herein in its entirety.

In particular embodiments, a ZSCAN4-inhibiting agent acts to inhibit telomerase, or prevent MRE11 from binding G-quadruplexes, and so forth. Therefore, in specific embodiments a ZSCAN4-inhibiting agent comprises more than one action (binding target through which it acts) that results in a therapeutic treatment or prevention of cancer.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, farnesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as Trays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

One or more immunotherapies may be employed with ZSCAN4-inhibiting agents for the treatment of benign or malignant tumors. The immunotherapy may encompass one of or both cell based and non-cell based methods. In specific embodiments, the immunotherapy utilizes checkpoint inhibition e.g. anti-PD-1/anti-PDL-1. In specific cases, the immunotherapies target PD-1 (Pembrolizumab or Nivolumab) or PD-L1 (Atezolizumab or Avelumab or Durvalumab) or CTLA-4 (Ipilimumab).

In some embodiments, immunotherapeutics utilize immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells, including those with chimeric antigen receptors that target a tumor antigen.

Immunotherapy, thus, could be used as part of a combined therapy, in conjunction with ZSCAN4-inhibiting agent(s). The general approach for combined therapy is discussed below. Generally, the tumor cell must bear some marker that is amenable to targeting, i.e., is not present on the majority of other cells. Many tumor markers exist and any of these may be suitable for targeting in the context of the present invention.

D. Genes

In yet another embodiment, the secondary treatment is a gene therapy in which a second therapeutic polynucleotide is administered before, after, or at the same time as ZSCAN4-inhibiting agent(s). Delivery of a vector encoding either an inhibitor of cellular proliferation and/or a regulator of programmed cell death may be utilized.

E. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

F. Other Agents

It is contemplated that other agents may be used in combination with the present disclosure to improve the therapeutic efficacy of treatment with ZSCAN4-inhibiting agent(s). These additional agents include immunomodulatory agents, agents that affect the upregulation of cell surface receptors and GAP junctions, cytostatic and differentiation agents, inhibitors of cell adhesion, and/or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abilities of the present invention by establishment of an autocrine or paracrine effect on hyperproliferative cells. Increases intercellular signaling by elevating the number of GAP junctions would increase the anti-hyperproliferative effects on the neighboring hyperproliferative cell population. In other embodiments, cytostatic or differentiation agents can be used in combination with the present invention to improve the anti-hyperproliferative efficacy of the treatments. Inhibitors of cell adhesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present disclosure to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

IV. Pharmaceutical Compositions

The term “pharmaceutical composition” relates to a composition for administration to an individual and encompasses one or more ZSCAN4-inhibiting agent(s).

In a particular embodiment, the pharmaceutical composition comprises a composition for parenteral, transdermal, intraluminal, intra-arterial, intrathecal or intravenous administration or for direct injection into a cancer. In one embodiment, the pharmaceutical composition is administered to the individual via infusion or injection. Administration of the suitable compositions may be effected by different ways, e.g., by intravenous, subcutaneous, intraperitoneal, intramuscular, topical or intradermal administration.

The pharmaceutical composition may further comprise a pharmaceutically acceptable carrier. Examples of suitable pharmaceutical carriers are well known in the art and include phosphate buffered saline solutions, water, emulsions, such as oil/water emulsions, various types of wetting agents, sterile solutions, etc. Compositions comprising such carriers can be formulated by well-known conventional methods. These pharmaceutical compositions can be administered to the subject at a suitable dose.

The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently.

The compositions of the disclosure may be administered locally or systemically. Administration will generally be parenteral, e.g., intravenous; DNA may also be administered directly to the target site, e.g., by biolistic delivery to an internal or external target site or by catheter to a site in an artery. In a preferred embodiment, the pharmaceutical composition is administered subcutaneously and in an even more preferred embodiment intravenously. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose), and the like. Preservatives and other additives may also be present such as, for example, antimicrobials, anti-oxidants, chelating agents, and inert gases and the like. In addition, the pharmaceutical composition of the present disclosure might comprise proteinaceous carriers, like, e.g., serum albumin or immunoglobulin, preferably of human origin. In certain embodiments, the pharmaceutical composition of the disclosure comprises, in addition to the proteinaceous chimeric cytokine receptor constructs or nucleic acid molecules or vectors encoding the same (as described in this disclosure), further biologically active agents, depending on the intended use of the pharmaceutical composition.

V. Kits of the Disclosure

Any of the compositions described herein may be comprised in a kit. In a non-limiting example, one or more ZSCAN4-inhibiting agents or one or more ZSCAN4 agonists may be comprised in a kit. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present disclosure also will typically include a means for containing the components in close confinement for commercial sale. Such containers may include injection or blow-molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, from which the formulation may be applied to an infected area of the body, injected into an animal, and/or even applied to and/or mixed with the other components of the kit.

However, the components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile, pharmaceutically acceptable buffer and/or other diluent.

In particular embodiments, ZSCAN4-inhibiting agent(s) one or more ZSCAN4 agonist(s) are provided in a kit, and in some cases the ZSCAN4-inhibiting agent(s) one or more ZSCAN4 agonist(s) are essentially the sole component of the kit. The kit may comprise instead or in addition reagents and materials to inhibit cell growth, such as cancer cell growth.

In particular embodiments, there are one or more apparatuses in the kit suitable for extracting one or more samples from an individual. The apparatus may be a syringe, scalpel, and so forth. In some embodiments, the kit includes one or more reagents or apparatus for diagnosis of cancer, including histological reagents, antibodies, blood or urine analysis reagents, and so forth.

In other embodiments, the kit, in addition to ZSCAN4-inhibiting agent(s) one or more ZSCAN4 agonists, also includes a second cancer therapy, such as chemotherapy, hormone therapy, and/or immunotherapy, for example. The kit(s) may be tailored to a particular cancer for an individual and comprise respective second cancer therapies for the individual.

### EXAMPLES

The following examples are included to demonstrate preferred embodiments of the invention. It should be appreciated by those of skill in the art that the techniques disclosed in the examples which follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

Telomeres are repetitive DNA sequences located at the ends of each chromosome that shorten with every cell division in order to regulate cellular aging, also known as senescence. As such, telomere shortening functions as a biological clock that limits the cells' ability to replicate indefinitely. As long as this clock is intact, replicating cells undergo senescence and stop dividing within approximately 40-60 cell divisions (Hayflick, et al., 1961). Conversely, cancer cells must overcome the process of aging in order to survive, replicate uncontrollably and form tumors (Gilley, et al., 2005; Lechel, et al., 2005; Yang, et al., 2008; Bryan, et al., 1997).

The ability to maintain telomere length in stem cells and cancer cells has traditionally been attributed to the enzyme telomerase (Kunicka, et al., 2008). It has been proposed that inhibition of telomerase will necessarily reduce cancer cell survival. However, other approaches are still required, as inhibition of telomerase can ultimately lead to resistance through other telomere maintenance mechanisms (Hu, et al., 2012), collectively called alternative lengthening of telomeres (ALT) (Bryan, et al., 1997; Hu, et al., 2012; Laud, et al., 2005).

ZSCAN4 (Zinc finger and SCAN domain containing 4) expression marks early preimplantation embryos and embryonic stem (ES) cells (Falco, et al., 2007). It has previously been shown that mouse ES cells maintain unlimited cell replications through activation of ZSCAN4, independent of telomerase (Zalzman, et al., 2010). The mouse ZSCAN4 was also shown to facilitate the nuclear reprogramming during the generation of induced pluripotent stem cells (iPSC) (Hirata, et al., 2012; Jiang, et al., 2013; Park, et al., 2015). Additionally, ZSCAN4 is involved in genomic stability and long term developmental potency of mouse ES cells (Amano, et al., 2013; Storm, et al., 2014; Akiyama, et al., 2015).

However, to date, little is known about the mechanism or function of the human gene ZSCAN4, and its significance to cancer remained unclear. The human and the mouse ZSCAN4 genes share 78% homology between their critical domains, suggesting they may serve similar functions.

In the present disclosure, the activity of the human ZSCAN4 and its contribution to cancer cell survival was determined. Remarkably, while the meta-analyses of ZSCAN4 expression in RNA-seq samples indicate it is expressed at low levels in some normal tissues, it is significantly upregulated across a spectrum of human cancers and cancer cell lines including head and neck, thyroid, lung, breast, skin, prostate, pancreatic and colon cancers. The study uncovers the novel role for human ZSCAN4 in telomere regulation by promoting DNA-RNA hybrids as well as recruiting the DNA recombination mediators MRE11A and stabilizing the complex with RAD50 at shortened telomeres, leading to telomere extension. Although ZSCAN4 coexists with telomerase in some cancers, it functions irrespective of telomerase activity. Consistent with these findings, ZSCAN4 depletion leads to a telomere mediated tissue culture crisis and severely inhibits tumor growth and development in vivo. The results demonstrate a critical role for ZSCAN4 in the maintenance of cancer tumor growth and survival and offer a useful new target for cancer therapeutics.

Examples of Results

Human ZSCAN4 is upregulated in cancer—One goal was to determine if ZSCAN4 is expressed in human cancer. In-silico analyses revealed that ZSCAN4 was upregulated in a range of human cancers including breast, cervical, lung (non-small cell), and head and neck cancer (Oncomine (Compendia, 2015; Rhodes, 2004), COSMIC (Forbes, et al., 2015)). Meta-analyses were further performed of ZSCAN4 expression using RNA-seq from cancer sample compared to normal organs and tumor adjacent tissues. The results indicate that ZSCAN4 is expressed at low levels in some normal tissues, yet its expression is significantly dysregulated (up or down-regulated) across a spectrum of human cancers and cancer cell lines including thyroid (63.7%), head and neck (42.4%), cervical (55.9%), and lung cancer (51.4%) (Table 1).

TABLE 1

| Cancer/ | ZSCAN4 | | | Tumor | |
|---|---|---|---|---|---|
| Tissue | Level | | | adjacent | |
| Type | (Rank) | Normal | Tumor | tissue | x² test |
| Thyroid | Upregulated | 17 | 299 | 5 | x²₍₄₎ = 294.96; |
| | Mid expression | 303 | 182 | 52 | p < 0.0001 |
| | Downregulated | 3 | 20 | 1 | |
| | Total no. of | 323 | 501 | 58 | |
| | samples | | | | |
| Head Neck | Upregulated | 7 | 131 | 14 | x²₍₄₎ = 200.2 |
| | Mid expression | 453 | 238 | 24 | p < 0.0001 |
| | Downregulated | 73 | 131 | 6 | |
| | Total no. of | 533 | 500 | 44 | |
| | samples | | | | |
| Cervix | Upregulated | 0 | 89 | — | x²₍₄₎ = 9.61; |
| | Mid expression | 9 | 134 | — | p < 0.05 |
| | Downregulated | 0 | 81 | — | |
| | Total no. of | 11 | 304 | — | |
| | samples | | | | |
| Lung | Upregulated | 9 | 185 | 25 | x²₍₄₎ = 161.86; |
| | Mid expression | 286 | 259 | 30 | p < 0.0001 |
| | Downregulated | 25 | 89 | 4 | |
| | Total no. of | 320 | 533 | 59 | |
| | samples | | | | |

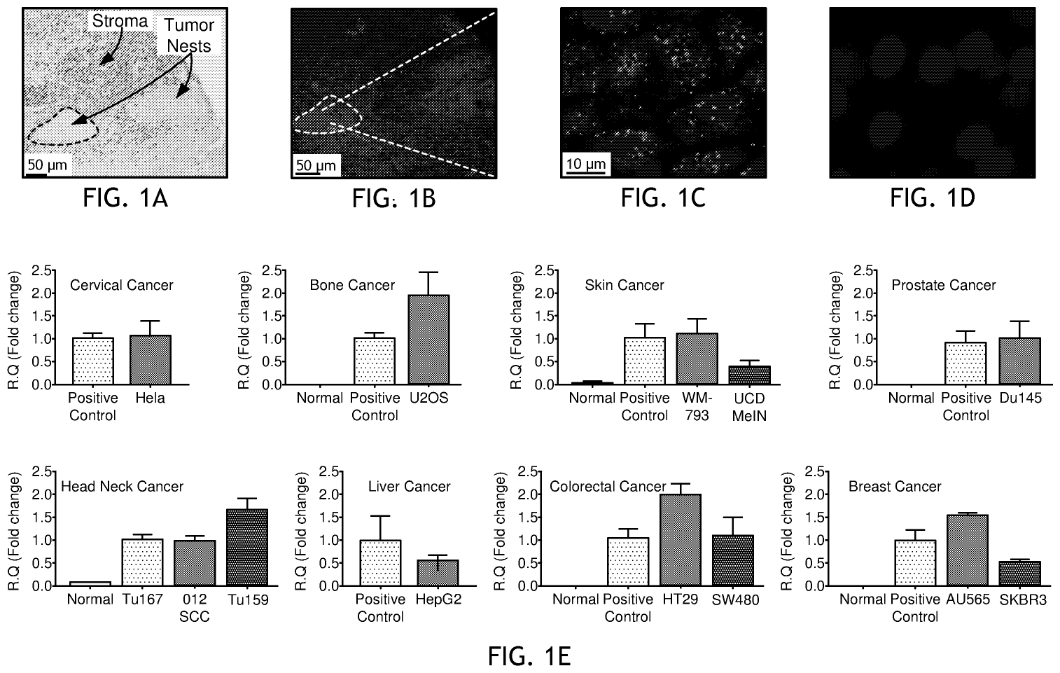

To validate ZSCAN4 protein expression in cancer, a human head and neck cancer tissue array was studied. The cancer tissue array included 118 cores derived from 46 cancer patients: 28 oral cavity cancer, 18 oropharynx cancer and 13 normal tissue controls. ZSCAN4 immunostaining followed by histological analysis in consecutive slices show positive staining in the tumor nests, but weak to no staining in the tumor stroma (FIG. 1A-C). The data further reveal that 100% of the intact oral cavity cancer cores and 67% of intact oropharynx cancer cores are positive for ZSCAN4, whereas normal tissues are negative (FIG. 1D). These findings indicate that ZSCAN4 expression may be restricted to the cancer cells within the tumor nests.

Another goal was to confirm ZSCAN4 expression in cancer cell lines in order to establish an in vitro model to perform gene function analyses. Therefore, ZSCAN4 expression was screened in multiple cancer cell lines by real time qPCR and by immunoblot analyses (FIG. 1E, 1F). ZSCAN4 is upregulated in the majority of the cancer cell lines tested, but not in normal cells (p≤0.001).

Collectively, the results indicate that while ZSCAN4 is not expressed in the normal tissues tested, it is upregulated across a spectrum of human cancer types, including at least head and neck, cervical, thyroid, lung, bone, skin, prostate, liver, colorectal and breast cancer (FIG. 1E, 1F).

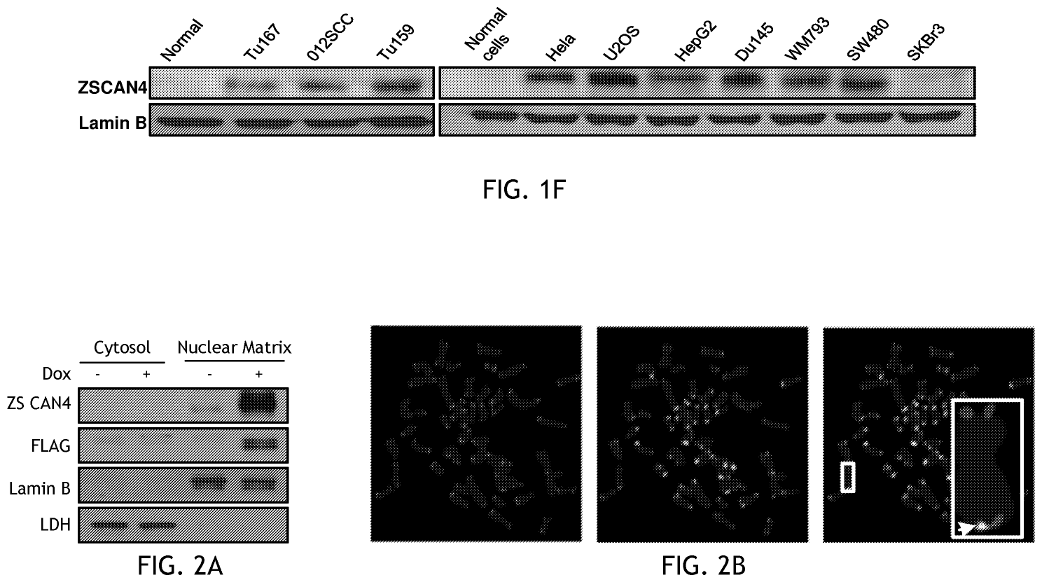

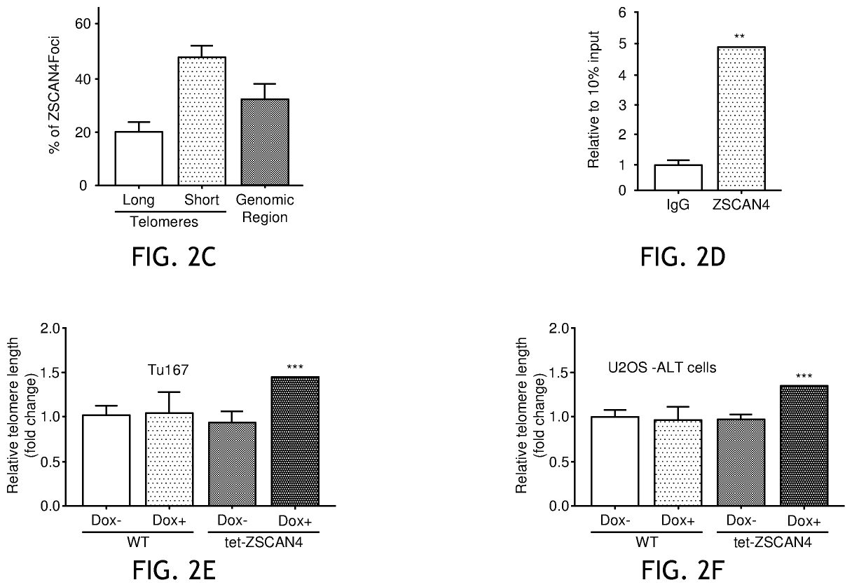

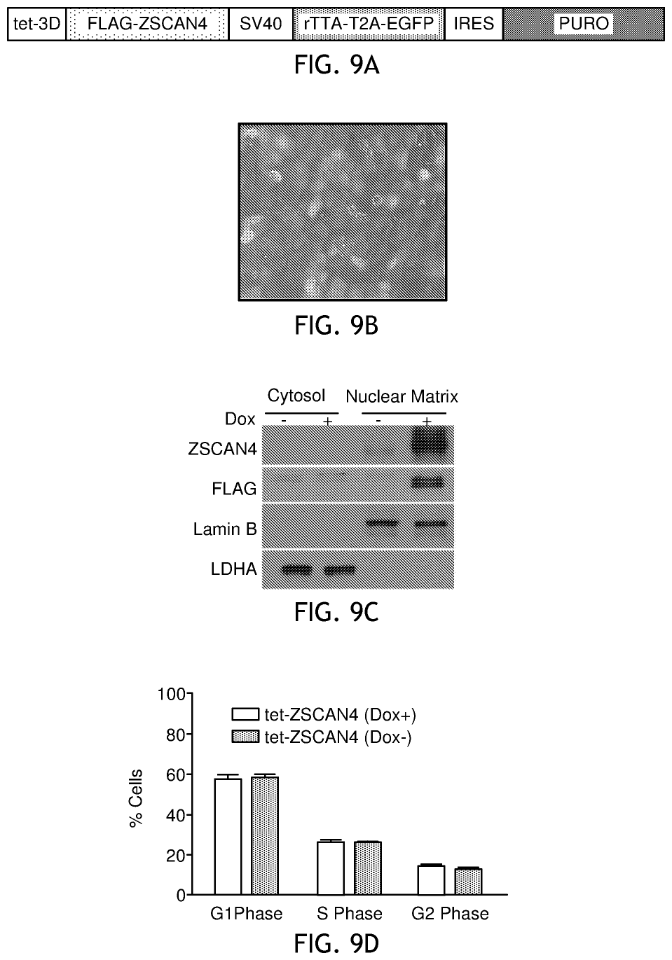

Human ZSCAN4 leads to telomere extension irrespective of telomerase activity—Ectopic expression of the human ZSCAN4 gene in mouse embryonic fibroblasts leads to higher efficiency in generation of induced pluripotent stem (iPS) cells (Hirata, et al., 2012), however, its effects on telomeres remained obscure. To further define ZSCAN4 function and its immediate effects in cancer cells, a doxycycline inducible tet-FLAG-ZSCAN4 lentiviral vector was designed. The vector includes a tetracycline transactivator (rTTA) fused to a GFP reporter gene and a puromycin selection gene (FIG. 9A, 9B). Inducible FLAG tagged ZSCAN4 cell lines (Tu167) were generated, named hereafter tet-FLAG-ZSCAN4. In these head and neck squamous cell carcinoma cell lines, ZSCAN4 is induced by addition of doxycycline (Dox), a tetracycline analog, to the culture medium. As expected, immunoblot analysis for ZSCAN4 or FLAG confirms ZSCAN4 induction in response to Dox treatment (FIG. 2A). No change or effect on cell cycle is observed following ZSCAN4 induction, suggesting it does not lead to immediate oncogenic effect (FIG. 9C).

Consistent with the attachment of telomeres to the nuclear matrix, fractionation of cellular protein content confirms FLAG-tagged ZSCAN4 is enriched in the nuclear matrix fraction (FIG. 2A). To define the interaction of ZSCAN4 protein with the telomeres, a co-localization study was performed by ZSCAN4 immunostaining along with telomere fluorescence in situ hybridization (T-FISH) in cancer cells (FIG. 2B). Co-localization analyses reveal that 70±0.9% (mean±S.E.M) of ZSCAN4 foci associate with telomeres, with significant preference for short telomeres (p≤0.01) (FIG. 2C). Moreover, chromatin immunoprecipitation (ChIP) assay with ZSCAN4 antibody followed by telomere qPCR indicate a significant interaction of ZSCAN4 with the telomeric repeats (p≤0.005) (FIG. 2D) compared to IgG control. Additional controls used: negative: Beta-actin; positive: RNA PolII IgG to demonstrate equal loading.

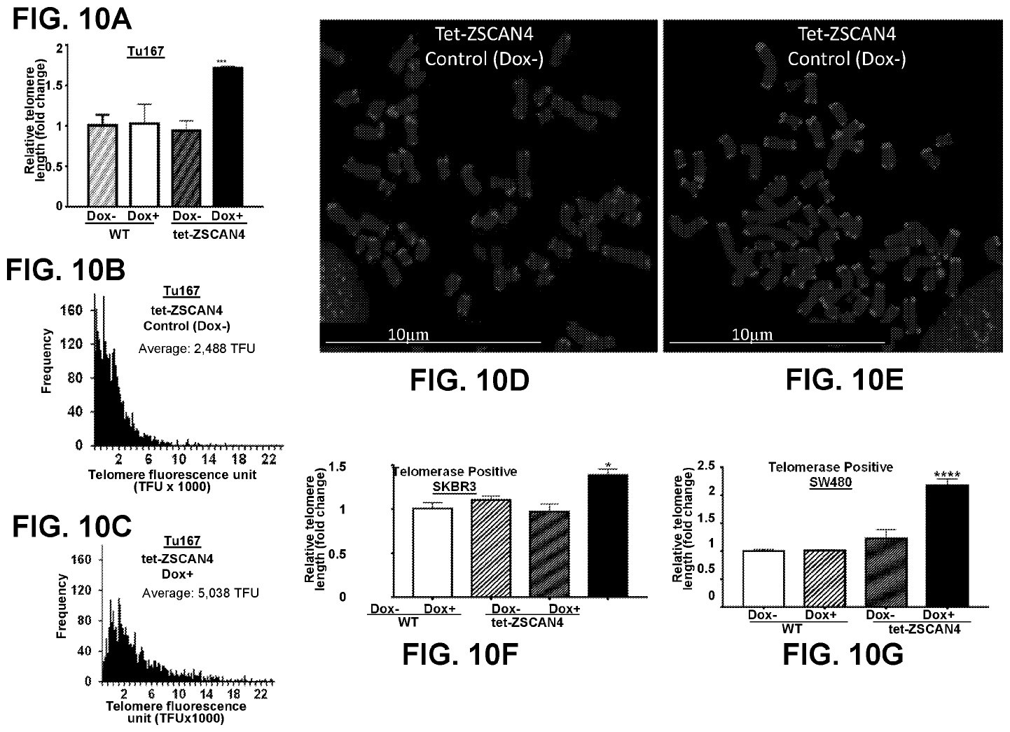

To find the effect of ZSCAN4 on telomere length, telomere real-time qPCR analyses were performed in which telomere length ratios were compared to a single-copy gene, as previously described (Zalzman, et al., 2010; Cawthon, et al., 2002; Callicott, et al., 2006). The findings show that ZSCAN4 leads to a significant 1.7±0.01-fold increase in telomere length (mean±S.E.M; p≤0.001), as measured by relative qPCR analysis (Tu167) (FIG. 2E). These results were confirmed by telomere quantitative FISH (Q-FISH (Zalzman, et al., 2010; Poon, et al., 1999) (FIG. 10) and independently demonstrated in additional two additional tet-FLAG-ZSCAN4 telomerase positive cell lines: breast (SKBR3) and colorectal cancer (SW480) (FIG. 10F, 10G). The results indicate that ZSCAN4 binds to telomeres of cancer cells and facilitates telomere extension.

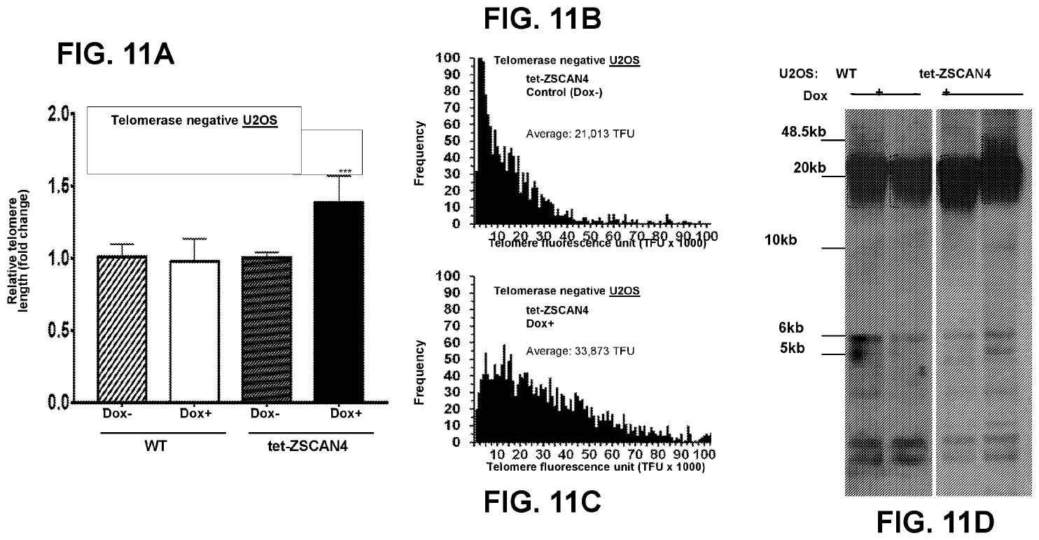

To find if ZSCAN4-mediated telomere elongation requires telomerase, Dox inducible tet-FLAG-ZSCAN4 cells in telomerase negative ALT cell lines (U2OS) were generated. The results demonstrate that ZSCAN4 induction for 48 hours leads to a significant telomere elongation as shown by telomere qPCR analysis (FIG. 2F), and is further confirmed by southern blot analysis. As expected, telomere length was not affected by Dox induction in the isogenic wild type (WT) U2OS control cells, as they show no significant difference in telomere length following Dox treatment (FIG. 9C). Consistently, the data indicate that the human ZSCAN4 mediated telomere extension is not associated with elevated telomerase activity in telomerase positive cells, as a mild reduction in telomerase activity was observed (FIG. 11B), nor is telomerase affected by ZSCAN4 depletion, as shown by Telomeric Repeat Amplification Protocol (TRAP) assay (FIG. 11C). The data indicate that ZSCAN4-mediated telomere extension does not require telomerase.

ZSCAN4 specifically binds and regulates TERRA DNA-RNA hybrids at telomeres—The preference of ZSCAN4 to short telomeres has prompted the inventors to study its possible involvement in telomere transcription. Previous reports have shown that short telomeres are transcriptionally active (Azzalin, et al., 2007; Schoeftner, et al., 2008) and express the long non-coding RNA, known as telomeric repeat-containing RNA (TERRA), which spans from the subtelomeres to the telomeric repeats (Azzalin, et al., 2007; Schoeftner, et al., 2008).

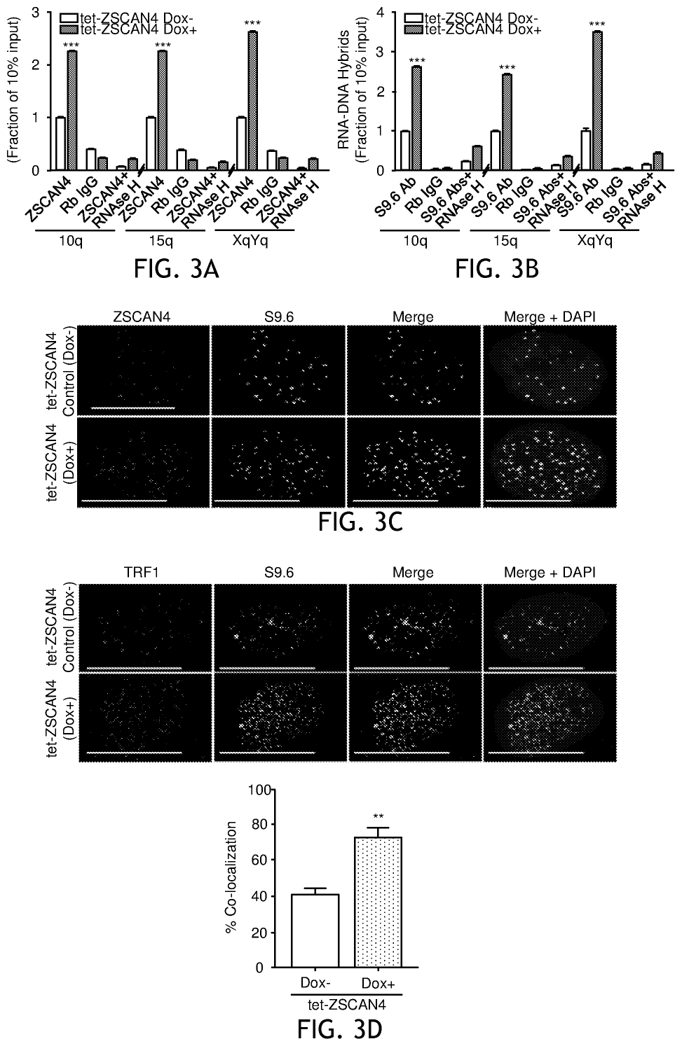

Like ZSCAN4, TERRA is able to localize to short telomeres (Deng, et al., 2009; Scheibe, et al., 2013). Subsequently, TERRA forms hybrids, and in the presence of recombination repair proteins, telomere length is modulated (Balk, et al., 2013; Yu, et al., 2014; Arora, et al., 2014). One goal was to examine the effect of ZSCAN4 on TERRA expression. The qRT-PCR analysis reveals ZSCAN4 induction has no significant direct effect on TERRA expression (FIG. 11A). To determine if ZSCAN4 can bind to TERRA chromatin or bind to telomeric DNA-RNA hybrids, the inventors performed chromatin immunoprecipitation (ChIP) analyses using ZSCAN4 antibodies and tested for TERRA, localized on the subtelomeric regions of chromosomes 10q, 15q and XqYq (Arora, et al., 2014; Arora, et al., 2012). The analyses indicate that ZSCAN4 binds to all subtelomeric TERRA regions tested. Furthermore, ZSCAN4 induction leads to a significant enrichment in ZSCAN4 binding of TERRA chromatin (p≤0.0001) (FIG. 3A). Notably, the results demonstrate that RNase H treatment, which selectively degrades DNA-RNA hybrids, abolishes ZSCAN4 binding to TERRA regions (FIG. 3A), indicating that without the RNA component ZSCAN4 was no longer enriched at these chromatin regions. These data indicate a specific interaction between ZSCAN4 and the DNA-RNA hybrids of TERRA and the telomeric DNA (p≤0.0001).

Next, to further define the interaction of ZSCAN4 with telomere DNA-RNA hybrids, DNA-RNA immunoprecipitation (DRIP) analysis was performed using an antibody (S9.6) that specifically recognizes DNA-RNA hybrid structures (Boguslawski, et al., 1986; Hu, et al., 2006). Consistent with previous findings, TERRA from all subtelomeric regions forms hybrids in both telomerase positive (Tu167) (FIG. 3B) and telomerase negative (U2OS) cells (FIG. 11B). Moreover, DRIP analyses following ZSCAN4 induction, demonstrates a significant increase (p≤0.0001) in TERRA DNA-RNA hybrids at telomeres in telomerase positive (FIG. 3B) and in telomerase negative cells (FIG. 11B) suggesting ZSCAN4 is involved in DNA-RNA hybrid regulation at telomeres independent of telomerase status.

The effects of ZSCAN4 are further demonstrated by co-localization analyses of ZSCAN4 with the DNA-RNA hybrid specific antibody S9.6. ZSCAN4 localizes to DNA-RNA hybrids (FIG. 3C) and the induction of ZSCAN4 increases the frequency of DNA-RNA hybrids. Additionally, to independently show the increase in DNA-RNA hybrids is at telomeres, co-immunostaining assays were performed using the S9.6 antibody and the telomere specific factor TRF1. The co-localization studies demonstrate that the vast majority of the DNA-RNA hybrids 71.4±6.12% (mean±S.E.M; p≤0.005) following ZSCAN4 induction are formed at telomeres (FIG. 3D). Taken together, the findings indicate ZSCAN4 as a novel regulator of DNA-RNA hybrids at telomeres.

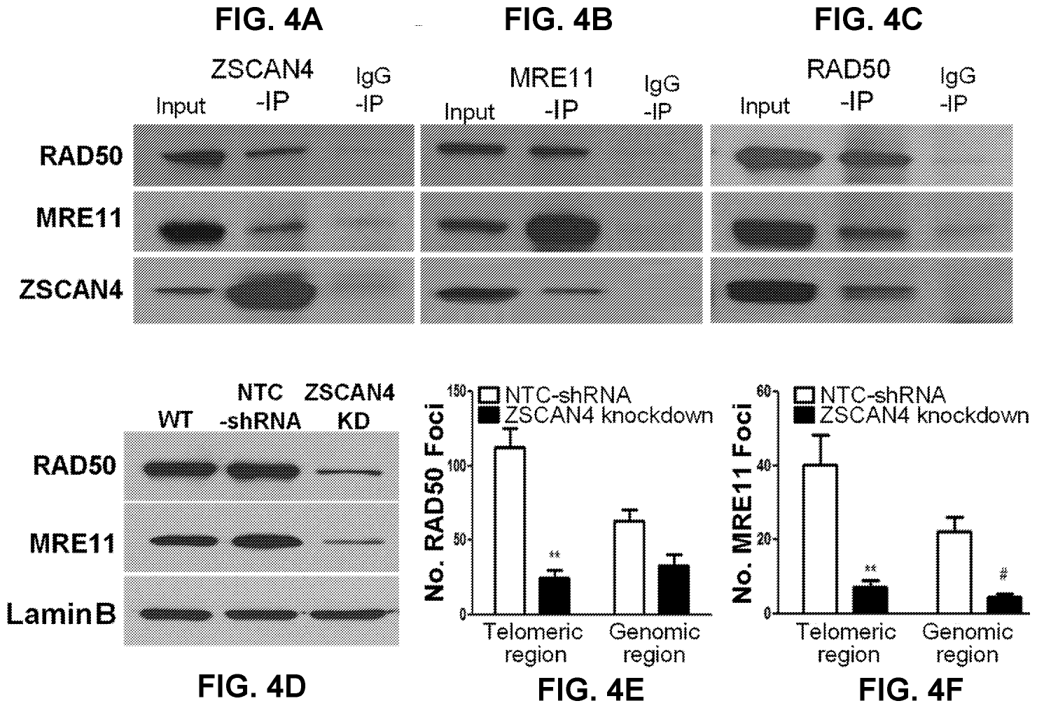

ZSCAN4 stabilizes recombination mediators on telomeres to promote telomere extension—The human ZSCAN4 protein contains a SCAN domain involved in protein-protein interactions, as well as four Zinc finger nucleic acid binding domains (Edelstein, et al., 2005), suggesting it may have a role in recruiting other factors to the chromatin. To further characterize the ZSCAN4 mechanism, the ZSCAN4 protein complex was studied by ZSCAN4 immunoprecipitation (IP) followed by mass spectrometry. At the top of the rank of interacting proteins, there were two major enzymes involved in recombination, MRE11A and RAD50, that form a complex with ZSCAN4. As a complex, these enzymes serve multiple functions at telomeres (Attwool, et al., 2009; Joseph, et al., 2010). To confirm the mass spectrometry data, the presence of RAD50 and MRE11A in the ZSCAN4 protein complex was confirmed by ZSCAN4 Co-IP followed by immunoblotting with RAD50 and MRE11A antibodies (FIG. 4A). Next, the inventors carried out a reverse-Co-IP with MRE11A (FIG. 4B) or RAD50 antibodies (FIG. 4C). Immunoblot analyses confirm these interactions as both RAD50 and MRE11 antibodies are able to efficiently pull down ZSCAN4. The results demonstrate that ZSCAN4 interacts with integral components of the DNA recombination machinery.

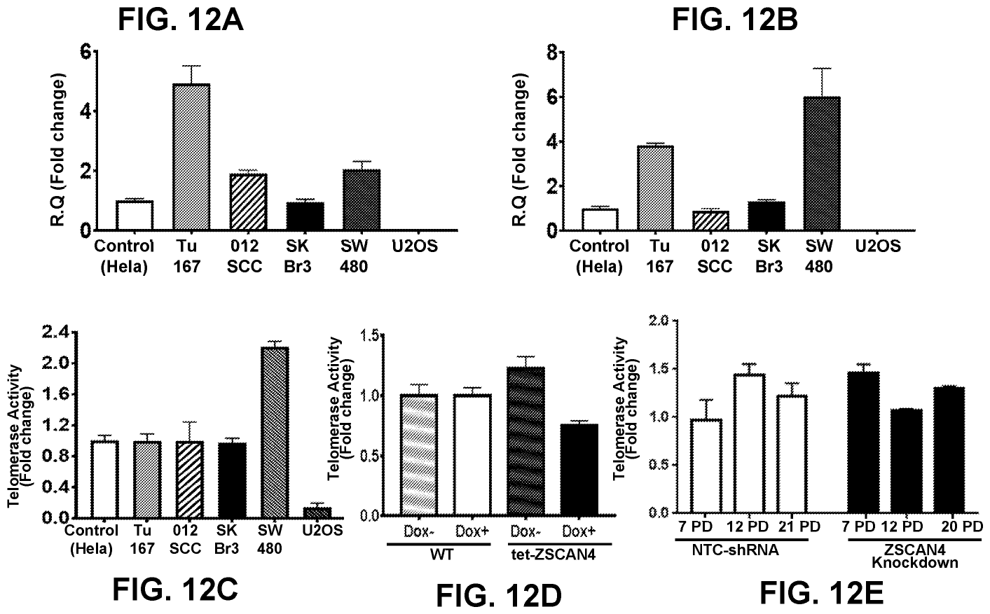

To further define the function of ZSCAN4 in the MRE11A/RAD50 complex, ZSCAN4 was inactivated by gene knockdown. Previous studies suggest that ZSCAN4 protein is indispensable for long term culture and telomere maintenance of mouse embryonic stem cells (Zalzman, et al., 2010), however, its role in cancer remained unknown. For that purpose, the knockdown efficiency was tested of four ZSCAN4 shRNA sequences (named shRNA1-shRNA4) by transfection into HNSCC cells (Tu167) (FIG. 12A). As controls, the inventors also generated isogenic lines with scrambled non-targeting control shRNA (NTC-shRNA) or an empty vector (Empty; the same vector without shRNA). The data by RT-qPCR, (FIG. 12B) and immunostaining analyses (FIG. 12C) confirm that all four ZSCAN4-shRNA sequences efficiently downregulate ZSCAN4 expression.

Following ZSCAN4 knockdown, immunoblot analyses indicate that a significant reduction in its interacting proteins RAD50 and MRE11 (FIG. 4D).

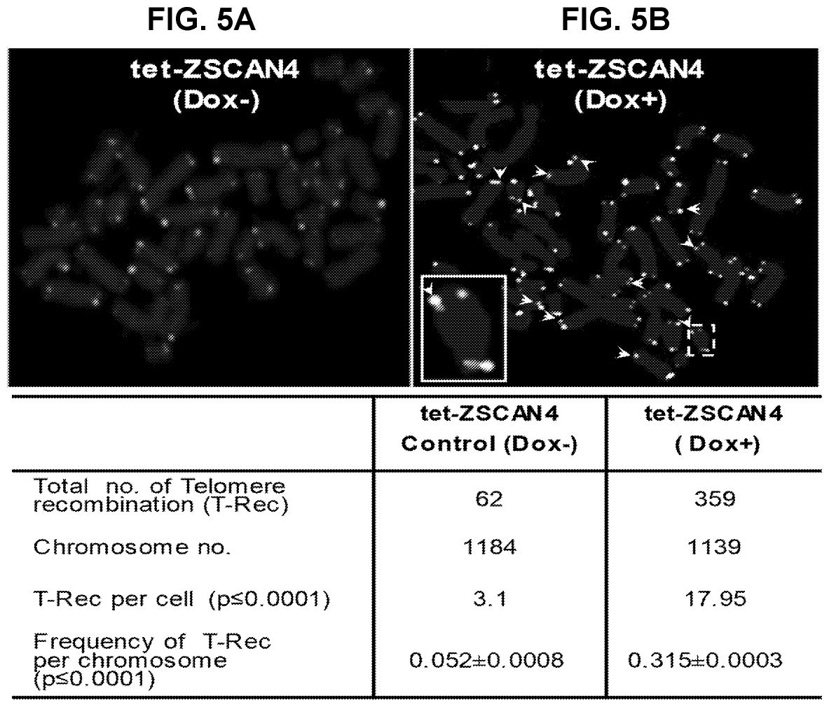

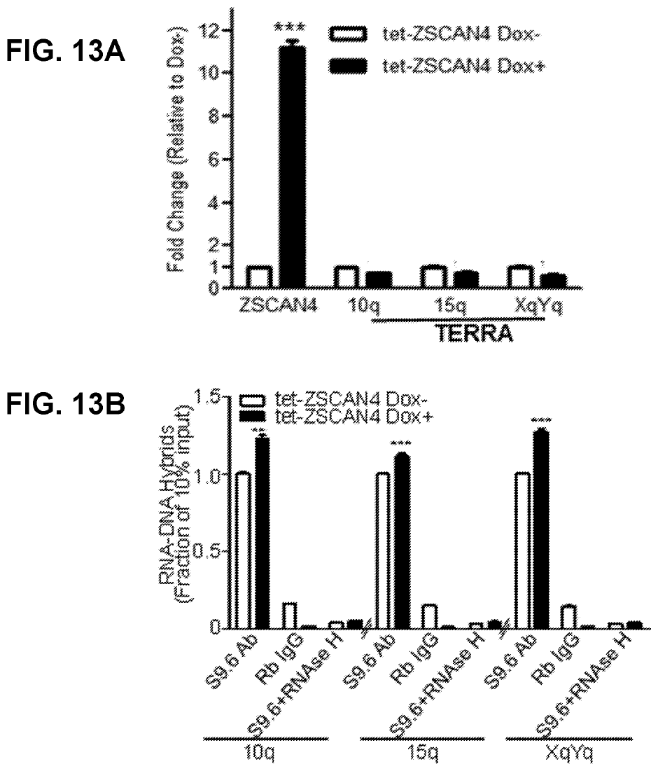

Next, co-localization analyses was performed of telomeres FISH with along with immunostaining of RAD50 or MRE11. The data confirm that loss of ZSCAN4 leads to a significant 3-fold reduction in the average number of RAD50 foci (FIG. 4E) and a 5.4-fold decrease in the average number of MRE11 foci (FIG. 4F). Furthermore, the data indicate that the major effect of loss of ZSCAN4 was on telomeric RAD50 and MRE11 foci while no significant effect was observed on the genomic foci of RAD50 and only a marginal effect on MRE11 genomic foci (P≤0.054) (FIG. 4E, 4F). Taken together and considering the effect of ZSCAN4 on telomere extension, these results suggests that ZSCAN4 stabilizes RAD50 and MRE11 at telomeres to mediate telomere recombination. Accordingly, it was important to define the effect of ZSCAN4 on telomeres recombination. Therefore, a telomere chromosome orientation FISH (CO-FISH) assay (Bailey, et al., 1996) was performed. ZSCAN4 induction leads to a significant, 6-fold increase (p≤0.0001) in the average frequency of telomere recombination events compared to non-induced controls (Dox−) (FIG. 5A, 5B; FIG. 13). Collectively, the findings suggest that ZSCAN4 binds and regulates DNA-RNA hybrids at telomeres and stabilizes a complex with MRE11A-RAD50 to mediate equal telomere recombination and thus extension.

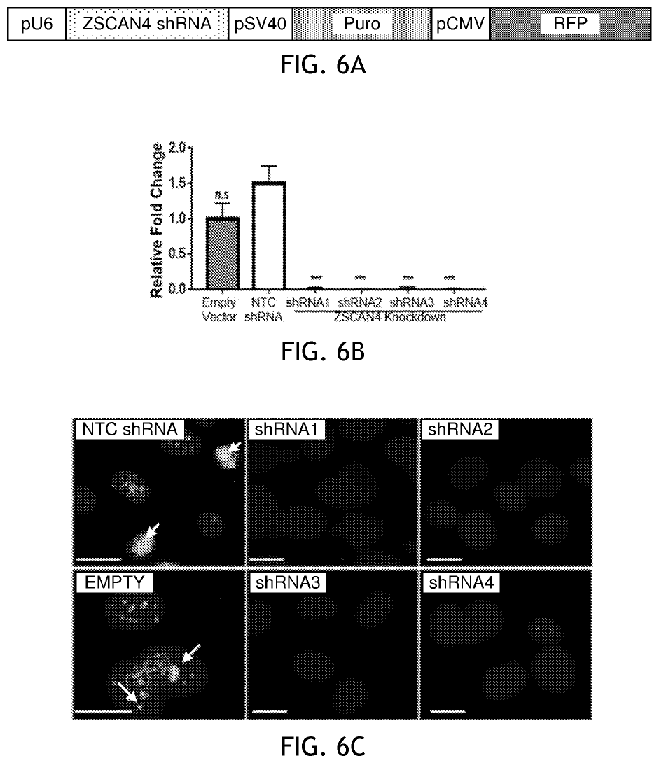

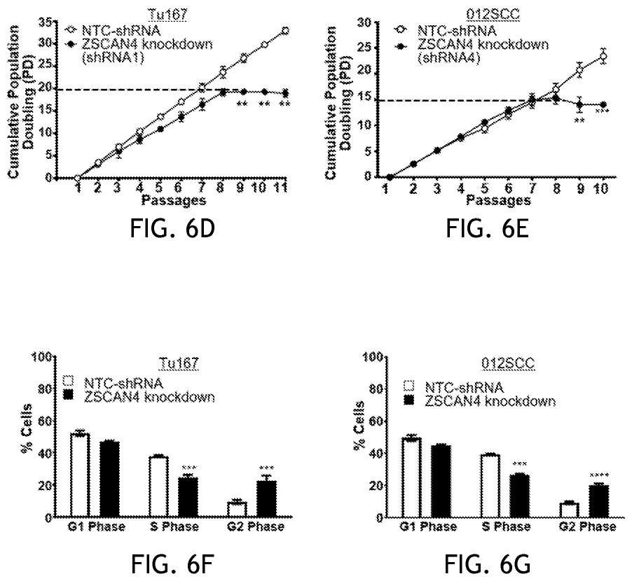

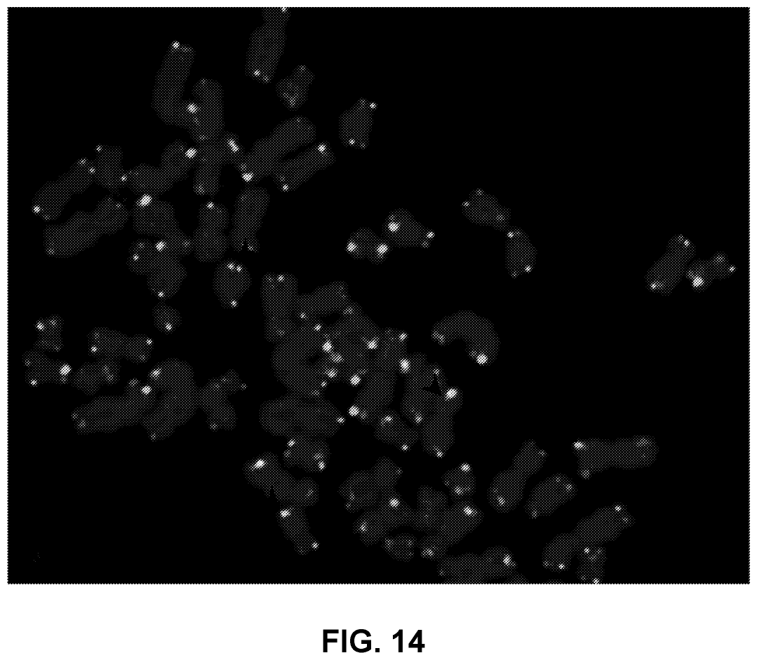

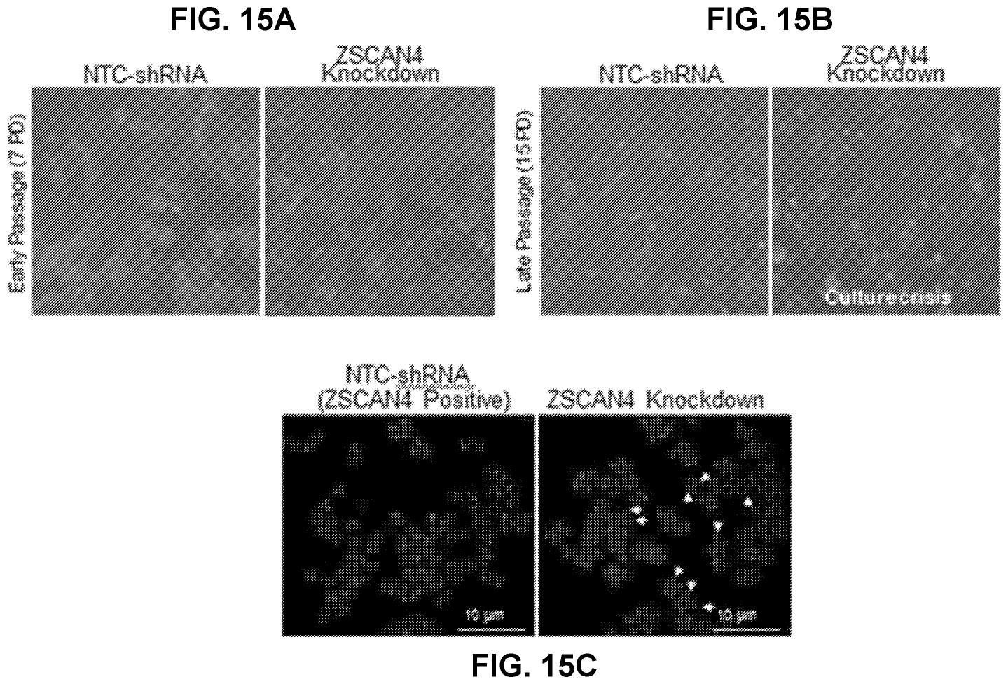

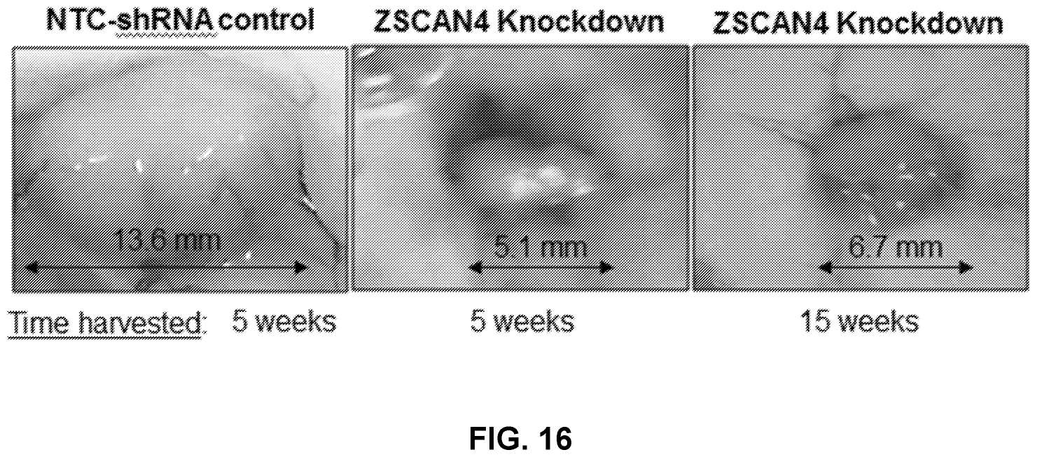

ZSCAN4 depletion leads to telomere shortening and culture crisis—Next, to directly study the effect of ZSCAN4 depletion in cancer cells, two independent, stable ZSCAN4 knockdown cell lines (Tu167 and 012SCC) were established. The results indicate that ZSCAN4 depleted cells initially proliferate and survive during cell expansion, similar to the control cells (FIG. 6A, 6B). However, the population doubling (PD) rate slows down significantly in both knockdown cell lines (FIG. 6A, 6B) by a total of 20 PDs in Tu167 (FIG. 6B) and 15 PDs 012SCC cells (FIG. 6B) whereupon cells ultimately reach culture crisis and abruptly die (FIG. 14A, 14B).

The observed reduction in population doubling capacity and culture crisis in ZSCAN4 depleted cancer cells prompted the inventors to investigate the effect on telomeres. Telomere qPCR analysis (Zalzman, et al., 2010; Cawthon, et al., 2002; Callicott, et al., 2006) was performed. Correlating to the reduced cell proliferation rate, the results show a gradual telomere shortening in ZSCAN4 depleted cells, and the average telomere length decreases significantly (p≤0.05). By passage 10, preceding culture crisis in both cell lines, telomeres become critically short (p≤0.001) (FIG. 6C, 6D). By contrast, telomeres of control cells remain at a constant length throughout all passages. These results show that loss of ZSCAN4 leads to gradual telomere shortening, reduced cell survival, and consequently to culture crisis.

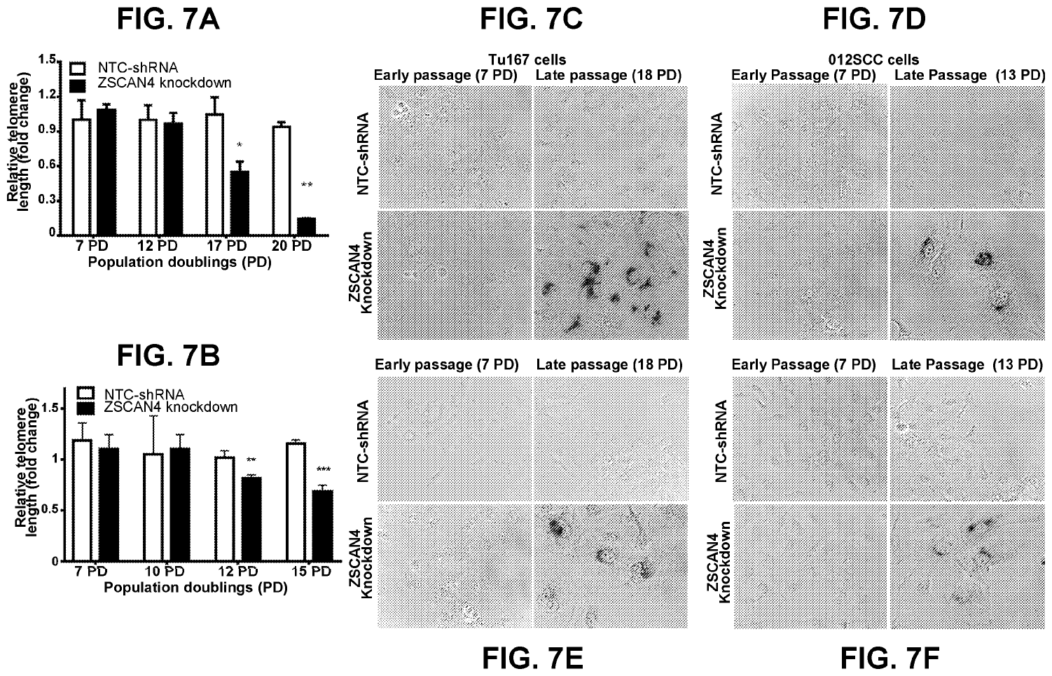

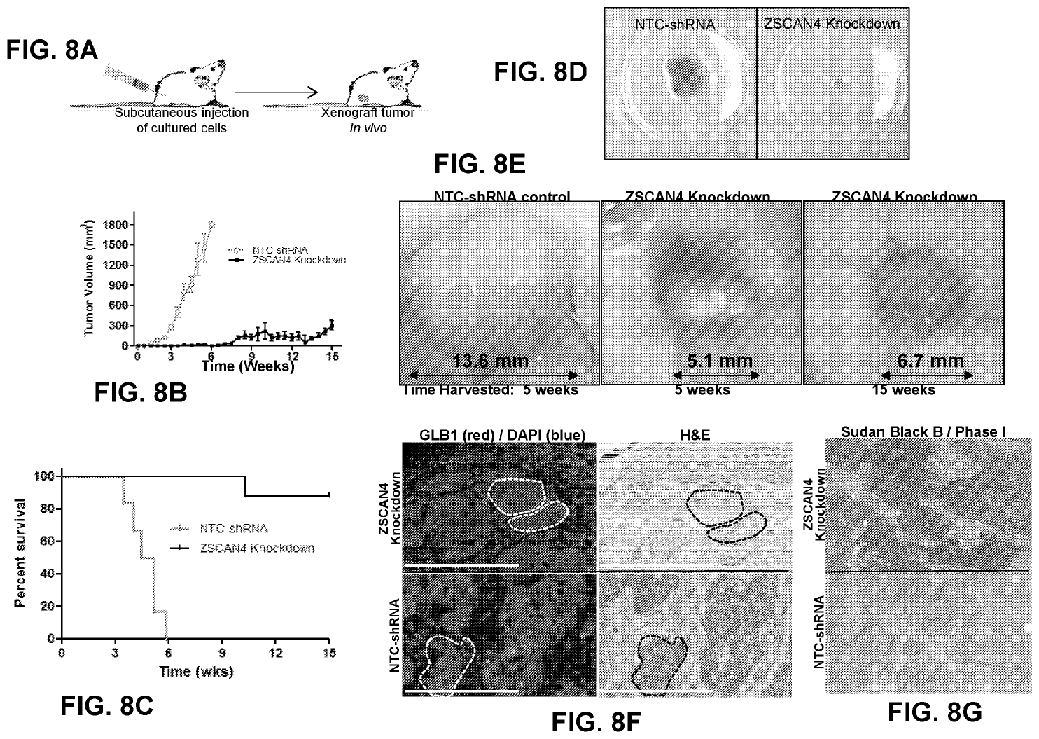

ZSCAN4 depletion severely affects tumor growth—To assess the utility of ZSCAN4 as a therapeutic target in cancer, the inventors monitored the impact of ZSCAN4 depletion on tumor growth in vivo using the NSG (NOD/SCID/IL2Ry−/−) (Ohbo, et al., 1996) mouse as a xenograft model. Either 1×10⁶ ZSCAN4 knockdown cells or 1×10⁶ non-targeting control (NTC) shRNA cells were subcutaneously injected into the flanks of female NSG mice (FIG. 7A). As expected, a large tumor develops in all mice injected with NTC-shRNA cells leading to euthanasia by week 5. Conversely, 90% of knockdown condition mice survive until the predetermined 15 week endpoint (FIG. 7B). Remarkably, the data indicate that ZSCAN4 depletion results in more than 98% tumor growth inhibition and only half of the mice present with a palpable tumor after 5 weeks. Furthermore, the tumors remain significantly attenuated even at the 15 week endpoint (FIG. 7C, 7D). At the end of the experiment, all tumors were taken for histological analyses and examined by an expert pathologist. ZSCAN4 depleted tumors harvested after 15 weeks show no significant increase in tumor grade over the duration of the experiment compared to both controls and ZSCAN4 knockdown tumors harvested after 5 weeks. The overall results suggest that ZSCAN4 depletion severely inhibits tumor growth and development in vivo.

Significance of Certain Embodiments

The ability to replicate indefinitely is ubiquitous to all cancer types and represents a major hallmark of cancer. Targeting cancer immortality by reinstating telomere shortening and thereby restricting tumor survival and growth offers an attractive strategy for cancer therapy. Therefore, understanding the underlying mechanisms that govern cancer cellular lifespan is crucial for potential new drug design and cancer prevention. Here it is shown that human ZSCAN4 is upregulated in a wide range of cancers and plays a critical role in the ability of cancer to defy cellular aging by facilitating rapid telomere extension.

While during embryonic development ZSCAN4 is expressed in early human preimplantation embryos (Yan, et al., 2013), and is downregulated thereafter, in human tissue, ZSCAN4 is expressed at low levels. One can characterize its activity in normal tissues and determine the cell types expressing ZSCAN4. Yet, it is upregulated across a spectrum of cancers. Interestingly, in mouse stem cells, reports have demonstrated that DNA damage (Storm, et al., 2014), telomere shortening (Nakai-Futatsugi, et al., 2016) and telomerase inactivation (Huang, et al., 2011) lead to ZSCAN4 upregulation. One can elucidate the signals leading to ZSCAN4 dysregulation in cancer.

Typically, ALT mechanisms are collectively thought to maintain telomere length in absence of telomerase activity. It is shown herein that human ZSCAN4 induction leads to telomere lengthening in telomerase negative and positive human cancer cells independent of telomerase status. Furthermore, ZSCAN4 complex analyses exclude a direct interaction of ZSCAN4 with telomerase or its complex components, and modulation of its expression does not affect telomerase activity. Taken together, these data indicate ZSCAN4-mediated telomere elongation in cancer does not require telomerase. This work has transformative implications, as it shifts the paradigm from two distinct mechanisms, into mechanisms that may coexist in ZSCAN4/telomerase positive cancer cells to regulate telomere length.

Remarkably, the studies uncover ZSCAN4 as the first positive regulator of DNA-RNA hybrid on telomeres. It is shown by ChIP and DRIP analyses that ZSCAN4 specifically binds TERRA hybrids at the telomeres irrespective of telomerase activity. Other previously reported factors are negative regulators of DNA-RNA hybrid, found to function specifically in telomerase negative ALT cells, such as the enzyme RNAseH1, which degrades DNA-RNA hybrids (Arora, et al., 2014; Balk, et al., 2013). Another factor, MORF4L2, affects TERRA levels both globally and at telomeres (Scheibe, et al., 2013), however, its effect on telomere length remains unclear. The study not only corroborates the programmed use of TERRA hybrids in the regulation of telomeres, but also defines ZSCAN4 as the first activator of this process irrespective of telomerase activity.

Previous reports in ALT cells show that increasing TERRA DNA-RNA hybrids contributes to telomere elongation by recombination. However, ALT cells presenting defective DNA recombination mechanism leads to rapid telomere shortening, most likely due to extensive replication fork stalling (Balk, et al., 2013). Therefore, a proper resolve of DNA-RNA hybrids at telomeres is essential for telomere maintenance. Remarkably, the data further show that ZSCAN4 forms a complex with the DNA recombination enzymes MRE11A and RAD50 on telomeres. The RAD50/MRE11A (Deng, et al., 2009; Dewar, et al., 2010; Haber, et al., 1998) complex has been shown to serve multiple functions at the telomeres. It degrades the telomere overhang to promote repair of dysfunctional telomeres (Attwooll, et al., 2009; Deng, et al., 2009; Dewar, et al., 2010; Joseph, et al., 2010; Lee, et al., 2003; Porro, et al., 2014). Further, MRE11A was shown to modulate telomeres by indirect interactions with TERRA (Porro, et al., 2014), yet the mechanisms controlling the recruitment of the complex to the telomeres are still not well understood. It is shown herein that ZSCAN4 induction lead to increased telomere recombination and extension. Overall, the results indicate that ZSCAN4 specifically binds DNA-RNA hybrid where it promotes equal telomere recombination and extension by forming a complex with MRE11A/RAD50 and enhancing their recruitment to telomeres.

Conversely, when ZSCAN4 is depleted by gene knockdown, the recruitment of MRE11/RAD50 complex to telomeres is significantly downregulated and the complex is disrupted, suggesting a role for ZSCAN4 in recruiting the recombination machinery to telomeres.

Consistent with these findings, ZSCAN4 depletion leads to a gradual telomere shortening, and ultimately to telomere-mediated culture crisis and the reinstatement of cellular aging in cancer cells. ZSCAN4 depletion abrogates tumor growth, indicating a critical role for ZSCAN4 in the maintenance of cancer cellular lifespan. Blocking the ZSCAN4 pathway offers new strategies for cancer prevention and means to restore cancer cellular aging in novel therapeutics.

Methods

Cell lines and cell culture. Head and neck squamous cell carcinoma (HNSCC) tumor cell lines TU167, TU159 and 012SCC were obtained from the University of Texas MD Anderson Cancer Center (Houston, Tex., USA) (Liu, et al., 1999). The cells used in our laboratory were originally generated by Dr. Gary Clayman and were reported to be free of cross contamination (Zhao et al., 2011). The cell line 012SCC was donated by Bert O'Malley from the University of Pennsylvania School of Medicine (Philadelphia, Pa., USA). All other cell lines were originally obtained from the ATCC. All cell lines were tested free of Mycoplasma. All tumor cell lines were cultured in complete DMEM medium (Invitrogen) supplemented with 10% fetal bovine serum (Atlanta Biologicals), 2 mM GlutaMAX, penicillin (100 U/mL), streptomycin (100 μg/mL).

Generation of ZSCAN4 knockdown and controls cells. Two HNSCC cells (Tu167, 012SCC) were transfected with 1 μg of pU6-ZSCAN4 shRNA vector (Origene) (containing RFP reporter and puromycin resistance gene), or controls: a non-targeting shRNA (NTC-shRNA) vector and an Empty vector (same plasmid without a shRNA cassette). Cells were transfected using Effectene reagent (QIAGEN) according to manufacturer's instructions. Cells were selected with 1 μg/ml Puromycin. Knockdown was confirmed by immunostaining and by qPCR.

Generation of tet-inducible ZSCAN4 vector and cell lines. The ORF of the human ZSCAN4 with an N-terminal FLAG-tag was cloned into the Lv208 lentiviral vector with Tet-3G inducible promoter, and SV40-rTTA-T2A-eGFP-IRES-puro cassette. The vector was co-transfected to generate lentiviral particles using Effectene (QIAGEN) according to manufacturer's protocol. Two cell lines (Tu167, 012SCC) were transduced and selected with puromycin thereafter named tet-FLAG-ZSCAN4 cells.

Generation of head and neck cancer tissue array. A tissue array was generated to include 118 cores derived from 46 cancer patients: 28 oral cavity cancer, 18 oropharynx cancer and 13 healthy controls (various sites) from the SOM/UMB Pathology Department tissue bank. Formalin-fixed paraffin-embedded tissue blocks were cut to 5 μm slices, stained by hematoxylin and eosin (H&E) and examined by an expert pathologist. Tumor was identified on the slide by the pathologist and the corresponding portion of the tissue block was targeted to cut a core for the array for each patient. The full array of cores was then embedded in paraffin and the block was used to cut 5 μm slices and prepare slides for histological analyses and immunostaining.

ZSCAN4 expression profiling in cancer. For ZSCAN4 gene expression profiling in cancer we used the TCGA database generated using RNA-seq for samples from breast, lung, cervical, head and neck, and thyroid cancers. Data was downloaded from the Genomic Data Commons Data Portal on the world wide web). To get gene expression for the corresponding normal tissue controls we downloaded the “Gene Read Count” data for breast mammary, lung, endocervix, exocervix, esophagus mucosa, esophagus muscularis, and thyroid tissues from the GTEx portal (on the world wide web) using the v6 release. These data were then further processed and analyzed using RStudio. To compare across samples, Gene expression levels (read counts) were normalized by calculating counts per million mapped reads (CPM) using the library size or the total number of reads per sample. Sample ranks of gene expression were used to compare across samples. We observed that the rank distributions for the data were typically bimodal in nature. Therefore, in order to compare the ranks across the three tissue types we classified samples rank as “downregulated” based on a floor value cutoff of <20,000 rank. Ranks above the mean rank plus one SD for normal tissue were considered as “upregulation” of expression. We then calculated the counts of samples for each of the tissue types and performed a Chi-squared test for significance in the difference of expression levels. Based on this test of significance we identified the following cancers where ZSCAN expression, as measured by the rank, was significantly upregulated.

Cancer Tissue array screen for ZSCAN4. Three consecutive slices of 5 μm were made of the tissue array for histological analysis by H&E, immunostaining and Masson trichrome. Expression of ZSCAN4 was studied using immunohistochemistry on consecutive sections of head and neck cancer tissue microarray containing oral cavity cancer samples as well as oropharynx cancer samples. Normal tissues were used as controls. Following deparaffinization with citrosolve solution, antigen retrieval was done with citrate buffer, pH=6 (sigma) staining was performed using the primary antibodies anti-ZSCAN4 (1:1000 dilution in a blocking solution) incubated overnight at 4° C. The bound antibody was visualized with a fluorescent Alexa546 secondary antibody (Invitrogen) under an EVOS FL microscope. Nuclei were visualized with DAPI (Roche).

Quantitative reverse transcription polymerase chain reaction (qRT-PCR). RNA was isolated and 1 μg of total RNA was reverse transcribed by Superscript III (Invitrogen) following the manufacturer's protocol. For qPCR, 10 ng cDNA was used per well in triplicates using SYBR green (Roche) following the manufacturer's protocol. Reactions were run on the LightCycler 480 system (Roche). Fold induction was calculated by the absolute quantification method. A standard curve was made for reference gene by serial dilutions of genomic DNA from 100 ng to 3.125 ng. The following primers were used: ZSCAN4 forward 5′-ATCCACCTGCCTTAGTCCAC-3′ (SEQ ID NO: 1) and ZSCAN4 reverse 5′-TCGAAGAACTGTTCCAGCCA-3′ (SEQ ID NO: 2), TERRA (Ohbo, et al., 1996; Arora, et al., 2012): chromosome 10q forward 5′-GAATCCTGCGCACCGAGAT-3′ (SEQ ID NO: 3) and chromosome 10q reverse 5′-CTGCACTTGAACCCTGCAATAC-3′ (SEQ ID NO: 4), TERRA chromosome 15q forward 5′-CAGCGAGAT TCTCC CAAGCTAAG-3′ (SEQ ID NO: 5) and TERRA chromosome 15q reverse 5′-AACCCTAACCACATGAGCAAC G-3′ (SEQ ID NO: 6), TERAA chromosome XqYq forward 5′-GGAAAGCAAAAGCCCCTCTGAAT-3′ (SEQ ID NO: 7) and TERRA chromosome XqYq reverse 5′-ACCCTCACCCTCACCCTAAGC-3′ (SEQ ID NO: 8), RPLP0 forward 5′-CAGCAAGTG GGAAGGTGTAATCC-3′ (SEQ ID NO: 9) and RPLP0 reverse 5′-CCCATTCTATCATCAACGGGTACAA-3′ (SEQ ID NO: 10), Telomere forward 5′-GGTTTTTGAGGGTGAGGGTGAGGGTGAGGGTGAGGGT-3′ (SEQ ID NO: 11) and Telomere reverse 5′-TCCCGACTATCCCTATCCCTATC CCTATCCCTATCCCTA-3′ (SEQ ID NO: 12).

Telomere Fluorescence In Situ Hybridization (T-FISH). All the cells were maintained in complete medium. Medium was replaced every 3 days. On the day of harvesting, medium was supplemented with 100 ng/ml colcemid (Invitrogen), followed by 6 hours incubation to arrest the cells in metaphase. After adding hypotonic 0.075 M KCl buffer, cells were fixed in cold methanol/acetic acid (3:1) and metaphase spreads were prepared. Telomere FISH was performed by Telomere peptide nucleic acid (PNA probe) (Bio-Synthesis). Chromosomes were stained with 1 μg/ml DAPI. Digital images of chromosomes and telomeres were captured by Zeiss microscope with Alexa546-DAPI filter sets.

Telomere Chromosome Orientation FISH (CO-FISH). CO-FISH analysis was done as previously described (Zalzman, et al., 2010; Bailey, et al., 1996; Goodwin, et al., 1993) with several minor modifications. Briefly, cells were incubated with 5′-bromo-2′-deoxyuridine (BrdU) for 12 hours to allow BrdU incorporation for one cell cycle. Colcemid (0.1 μg/ml) was added for the final 6 hours. Metaphase spreads were prepared. Slides were stained with 0.5 μg/ml Hoechst 33258 (Sigma), washed in 2×SSC for 20 min at room temperature, mounted with 2×SSC buffer (at pH 8.0), and exposed for 30 min to a 365-nm UV light at a distance of 10 cm away from the bulb (Stratalinker 1800 UV Irradiator). The BrdU-substituted DNA was digested with 3 units/μl Exonuclease III (Promega) for 10 min at room temperature. The leading strand telomeres were revealed by 5′ Alexa546-TTAGGGTTAGGGTTAGGG 3′DNA probe (IDT) (SEQ ID NO: 13) without denaturation step and incubated 2 hours at room temperature. Slides were washed and the lagging strand telomeres were revealed by a PNA probe 5′Alexa488-OO-CCCTAACCCTAACCCTAA (Bio-Synthesis) (SEQ ID NO: 14) without denaturation step and incubated 2 hours at room temperature. Chromosomes were counterstained with 1 μg/ml DAPI (Vector Laboratories). Telomere recombination events were counted n≥1139; N=no. of chromosomes per sample from at least two independent experiments. Data were analyzed by using two-tailed unpaired Student's t-test.

Telomere measurement by quantitative real-time PCR. Genomic DNA was extracted from 10⁶ cells and quantified by Nanodrop. Average telomere length ratio was measured from total genomic DNA using a real-time PCR assay as previously described (Zalzman, et al., 2010; Cawthon, et al., 2002; Callicott, et al., 2006). Briefly, qPCR was performed on the LightCycler 480 system (Roche), using telomere primers: hTELO-F: GGTTTTTGAGGGTGAGGGTGAGGGTGAGGGT GAGGGT (SEQ ID NO: 15), hTELO-R: TCCCGACTATC CCTATCCCTATCCCTATCCCTATCCCTA (SEQ ID NO: 16). A standard curve was made for reference gene by serial dilutions of genomic DNA from 100 ng to 3.125 ng. The telomere signal was normalized to the single copy gene RPLP0 to generate a telomere vs. single copy (T/S) gene ratio, indicative of relative telomere length. Results are shown as mean±S.E.M in three biological replicates. These observations were demonstrated repeatedly in multiple independent experiments. Data were analyzed by two-way ANOVA with repeated measures and Tukey's-tests.

Co-immunohistochemistry with Telomere FISH. High-quality metaphase spreads were prepared and stained as previously described (Zalzman, et al., 2010). Briefly, slides were unmasked in buffer citrate at 90° C., dehydrated, and incubated for 5 min at 87° C. with Alexa488-conjugated Telomere PNA probe (AF488-OO-CCCTAACCCTAACCCTAA) (Bio-Synthesis) (SEQ ID NO: 17) or with Alexa568-conjugated DNA probe TTAGGGTTAGGGTTAGGG/3AlexF594N/(IDT) (SEQ ID NO: 18). Slides were allowed to anneal at room temperature for 1 hour. Primary antibodies were diluted in block solution as follows: mouse anti-ZSCAN4 (1:1000) (Origene), rabbit anti-MRE11A (1:400) (Cell Signaling) and incubated overnight at 4° C. Slides were incubated for 1 hour at room temperature with secondary antibodies (Invitrogen) (diluted in block solution): Alexa 568 Donkey anti Rabbit (1:800) or Alexa 568 Donkey anti mouse (1:800). Nuclei were counterstained with DAPI. Cells were visualized by Zeiss 510-confocal microscope followed by co-localization study was performed by ImageJ software (Schneider, et al., 2012) per each condition; results are shown as mean±S.E.M. Data were analyzed by one-way ANOVAs and Tukey's test.

Immunoblot analysis. All cancer cell lines were grown in 15 cm plates. Nuclear proteins were fractionated using cytoskeleton buffer (10 mM PIPES, 300 mM Sucrose, 100 mM NaCl, 3 mM MgCl₂, 1 mM EGTA. 0.5% Triton X100), lysed in urea (8 M Urea solution in 0.01 Tris pH 8+0.1 M NaH₂PO₄), sonicated and quantified. 100 μg of each sample was loaded on 8% SDS-PAGE and immunoblotted for endogenous ZSCAN4. tet-FLAG-ZSCAN4 HNSCC Tu167 cells were allowed to form a monolayer in a 10 cm plate and to induce ZSCAN4 expression with Dox. Nuclear proteins were isolated using Nuclear extract kit (Active Motif, CA). 30 μg of nuclear lysates were separated on SDS-PAGE, transferred to PVDF membrane and incubated with the primary antibody over night at 4° C. Antibodies used: anti-ZSCAN4 1:1000 (Origene), anti-MRE11A (Cell Signaling) 1:1000, anti-RAD50 (Cell Signaling) 1:1000. Membranes were then incubated with secondary antibodies conjugated with HRP (horseradish peroxidase) (Millipore), and washed with TBST. Protein bands were detected using chemiluminescence detection system (ThermoScientific). All immunoblots shown represent at least 3 independent experiments.

Telomerase activity measurement. All the cells in different passages were cultured in triplicate in complete medium and harvested after 2 days. Cell lysates were prepared from 10⁶ cells per sample. Telomerase activity was measured by TRAP assay using a TRAPEZE Telomerase Detection Kit (Millipore) according to the manufacturer's instructions. Positive controls: isogenic NTC-shRNA cells in equivalent passages, isogenic WT cell extract and telomerase positive HeLa cells. Technical negative controls used: heat inactivated extracts per each sample. Results are shown as mean±S.E.M, in three biological replicate obtained from 3 independent experiments. Data were analyzed by two-way ANOVA.

Immunohistochemistry. Cells were either fixed in 4% PFA for 10 min at room temperature or taken for metaphase spreads as described above. Cells in PFA were permeabilized with 0.2% NP-40 for 10 min. Cells were blocked for 10 min at room temperature in 1% BSA, 10% fetal bovine serum, and 0.2% Tween 20 and incubated overnight at 4° C. with the primary antibodies in a blocking solution: a Primary antibodies were incubated over night at the following dilutions: mouse anti-ZSCAN4 (1:1000), rabbit anti-MRE11A (1:400). Slides were incubated for 1 hour at room temperature with secondary antibodies (diluted in block solution): Alexa 488 Donkey anti Rabbit (1:400); Alexa568 Donkey anti mouse (1:800). Nuclei were stained with DAPI for 10 min at room temperature. Scramble non-targeting shRNA cells and empty vector cells were used as positive controls. Additional controls were cells stained without primary antibody. The bound antibody was visualized with a fluorescent Alexa546 secondary antibody (Invitrogen) under a Zeiss 510-confocal microscope. Nuclei were visualized with DAPI (Roche Life Sciences). Quantification of co-localization was visualized by Zeiss 510-confocal microscope and co-localization was performed by ImageJ software per condition; results are shown as mean±S.E.M Data were analyzed by T-test.

Tumor histological analysis. Degree of intra-tumoral inflammation assessment was done as double blind assay by the expert pathologist according to the following guidelines: Area=0.0385 mm² (40×); Area of 10 fields=0.4 mm². Degree of Apoptosis was measured in area of greatest apoptosis. HPF=40×. The number of Mitoses was measured in an area of greatest mitotic activity of ×10 microscope field.

Terminal restriction fragment length southern blot analysis. Telomere southern blot analysis was done as previously described. Briefly, to prepare Genomic DNA, 3×10⁶ cells were lysed in SDS Buffer (0.5% SDS diluted in 200 mM Tris (pH 8.1), 25 mM EDTA, 250 mM NaCl) and treated with 10 uL of RNAse H (20 mg/mL) followed by 10 uL proteinase K (10 mg/mL). DNA was extracted with phenol chloroform and precipitated with isopropanol. Terminal DNA fragments were generated from 5 ug of genomic DNA by HinfI and RsaI restriction enzyme digestion, electrophoresed through a 0.5% agarose gel, and transferred to a membrane (Hybond-N+; Amersham). DNA bands were detected by hybridization with a 3′ biotin-labeled probe (CCCTAACCCTAACCCTAA) (IDT) (SEQ ID NO: 19), crosslinked to the membrane with UV at 1200 joules (Stratalinker 1800 UV Irradiator) and visualized using a Chemiluminescent Nucleic Acid Detection Kit (Pierce) according to the manufacturer protocol.

Cell cycle assay. A monolayer of the stable cells were treated without or with 1 μg/ml doxycycline for 24 hours, harvested by accutase, washed twice with cold PBS, and centrifuged at 500 g for 5 min. Then, 5×10⁵ cells were resuspended in 0.2 ml 1×PBS and 1.4 mL 80% ice cold ethanol was added dropwise and incubated on ice for 2 hours. Cells were washed once with 1 mL PBS and resuspended in 200 μL PBS and incubated with RNase A for 5 min at room temperature, and stained with Propidium Iodide (PI) and assessed by flow cytometer on SSC pulse width (SSC-W) and analyzed by FlowJo software. n=8 per group of biological replicates from multiple independent experiments. Data were analyzed by two tailed, unpaired Student's t-test.

Co-immunoprecipitation. Protein A/G beads (25 μL) were incubated with 1 μL primary antibody in 100 μL IP-RIPA buffer (RIPA buffer without SDS) on a rotating plate for 1 h at room temperature. Then, the beads were centrifuged at 1000×g for 2 min at 4° C. The supernatant was discarded and the beads were washed with IP-RIPA buffer. Beads were resuspended in 100 μL of 5 mM BS3 solution and incubated at room temperature for 45 min with rotation in order to cross-link antibody to the beads. After washing, the beads were incubated with 100 μL of pre-cleared nuclear lysate and incubated overnight at 4° C. Protein bound antibody cross-linked with protein A/G was centrifuged and washed. Immunoprecipitated proteins were eluted with 25 μL RIPA with 0.1% SDS and 25 μL of 2× loading dye. 25 μL of the eluted proteins were subjected to SDS-PAGE and immunoblotted.

Chromatin immunoprecipitation (ChIP) analysis. To study the association of ZSCAN4 with the telomere or with TERRA, tet-FLAG-ZSCAN4-Tu167 cells were grown to 50% confluency and incubated for 48 hours with 1 μg/ml doxycycline. Cross-linking and chromatin immunoprecipitation was done using the Pierce Magnetic ChIP Kit (Thermo Scientific) according to the manufacturer's instructions with the following modifications. Samples were preheated to 65° C. for 20 minutes, followed by treatment in the presence or absence of RNase H (100 U) at 37° C. for 16 hrs. RNAse H was inactivated and samples were taken to ChIP assay. 10% of the input of each sample was saved for normalization purposes. Anti-ZSCAN4 (10 μg) (Origene) was used for immunoprecipitations, and the provided anti-RNA Polymerase II (1 μg) and Rabbit IgG (10 μg) were used as controls. Next samples were incubated in 20 μl of Protein A/G Magnetic beads (Pierce) according to the manufacturer protocols. Beads were washed and samples were eluted following Pierce Magnetic ChIP Kit protocol. Nucleic acids were purified using a PCR Clean Up Kit (Qiagen). Purified DNA was taken for real time qPCR using the same methods and primers for TERRA described in the qRT-PCR method. Data are shown as mean±S.E.M from 3 independent experiments. Data were analyzed using two-way ANOVAs with repeated measures followed by Tukey's posthoc comparison tests were used for statistical analyses.

DNA immunoprecipitation (DRIP) assay. DNA Immunoprecipitation was performed as previously described (Arora, et al., 2014) with the following modifications: Cells were harvested on ice and lysed in 1 ml of RA1 buffer (Macherey-Nagel) containing 10 μl of β-mercaptoethanol. Samples were then either treated with Proteinase K (Qiagen). DNA samples were purified with phenol: chloroform: isoamyl alcohol (25:24:1 saturated with 10 mM Tris-Cl pH 8.0, 1 mM EDTA), and precipitated in isopropanol. DNA pellets were re-suspended in Tris-EDTA and sonicated with a 550 Sonic Dismembrator (Fisher Scientific) to obtain 100-500 bp long fragments. 10 ug of sheared nucleic acids were diluted in 1 ml of IP buffer (0.1% SDS, 1% Triton X-100, 10 mM HEPES pH 7.7, 0.1% sodium deoxycholate, 275 mM NaCl). DNA samples were treated (or remained untreated in buffer only), with 100 U of RNaseH (NEB) for 16 h at 37° C. prior to IR For DNA IP, samples were incubated for 2 h on a rotating wheel at 4° C. in presence of 5 μg of S9.6 antibody or with Rabbit IgG (10 μg) were used as controls. Next, samples were incubated in 20 μl of Protein A/G Magnetic beads (Pierce) according to the manufacturer protocols. Beads were washed and samples were eluted following Pierce Magnetic ChIP Kit protocol. Purified DNA was eluted and Nucleic acids were purified using a PCR Clean Up Kit (Qiagen). Real time qPCR was performed using the same methods and primers for TERRA described in the qRT-PCR method. Data are shown as mean±S.E.M from 3 independent experiments. Data were analyzed using two-way ANOVAs with repeated measures followed by Tukey's posthoc comparison tests were used for statistical analyses.

Statistical analyses. Results are shown as the mean±S.E.M of multiple independent experiments, with biological replicates. Detailed n values for each panel in the figures are stated in the corresponding Methods section. Student's t-test, one-way or two-way ANOVAs with repeated measures followed by Tukey's posthoc comparison tests (when appropriate) were used for statistical analyses. Survival curves were derived from Kaplan-Meier estimates using Log-rank (Mantel-Cox) test. All statistical analyses were performed with STATISTICA 12 and GraphPad Prism 5 software. All statistical tests were two-tailed and P values <0.05 were considered to be statistically significant.

For in vitro experiments, sample size was determined based on review of the literature as well as previous experience in the laboratory. The investigators were not blinded to allocation during experiments and outcome assessment. For in vivo experiments, sample size and power of the test calculations were done using our preliminary data based on pilot experiments. Power analysis was based on a several plausible differences (delta) in the time-averaged tumor size between two treatment groups, as described by Diggle et al. (1996). The mice were randomly assigned to the experimental groups. Investigators were blinded to experimental groups and outcome assessments during experiments.

### Example 2

### Zscan4 is Negatively Regulated by the Ubiquitin-Proteasome System

Zscan4 is an early embryonic gene cluster expressed in mouse embryonic stem and induced pluripotent stem cells where it plays critical roles in genomic stability, telomere maintenance, and pluripotency. Zscan4 expression is transient, and characterized by infrequent high expression peaks that are quickly down-regulated, suggesting its expression is tightly controlled. However, little is known about the protein degradation pathway responsible for regulating the human ZSCAN4 protein levels. In this study we determine for the first time the ZSCAN4 protein half-life and degradation pathway, including key factors involved in the process, responsible for the regulation of ZSCAN4 stability. It is demonstrated that lysine 48 specific polyubiquitination and subsequent proteasome dependent degradation of ZSCAN4, which may explain how this key factor is efficiently cleared from the cells.

### INTRODUCTION

The embryonic gene Zscan4 (Zinc finger and SCAN domain containing 4) promotes telomere and genomic stability in mouse embryonic stem (ES) cells (Zalzman et al., 2010). Knockdown of Zscan4 in mouse ES cells results in telomere shortening and karyotype abnormalities, slowing cell proliferation until reaching culture crisis. Zscan4 is highly but transiently expressed (Zalzman et al., 2010), with protein expression bursts that facilitate chromatin remodeling (Akiyama et al., 2015; Amano et al., 2013) and transcriptional reprogramming during the generation of induced pluripotent stem (iPS) cells (Hirata et al., 2012; Jiang et al., 2013; Park et al., 2015). A short expression burst of Zscan4 was further demonstrated to replace Myc and enhance the efficiency of mouse iPS cell formation through activation of early embryonic genes (Hirata et al., 2012). The human ZSCAN4 has been shown to interact with factors important for telomere maintenance (Lee et al., 2015; Lee et al., 2014), and has been suggested to play a role in cancer (Zalzman et a., 2010; Lee et al., 2014). Given the important role of ZSCAN4 and its transitory nature in the cell (Zalzman et al., 2010; Falco et al., 2007), maintaining the delicate balance between its protein synthesis and degradation is critical for stem cell and potentially cancer cell function.

Concentrations and spatial gradients of specific proteins must be able to rapidly change in response to extracellular cues and according to current cell state (Korolchuk et al., 2010). Small protein imbalances can drastically impact such important cellular processes. Therefore, intracellular protein degradation and turnover play a significant role in cell life cycle (Ciechanover et al., 2005). Two major pathways responsible for the degradation of proteins in cells are autophagy and the ubiquitin proteasome system (UPS). Autophagy is the process responsible for degradation of longer lived, structural proteins and organelles. This process depends on the formation of a double membrane autophagosome, which takes up its cargo and subsequently fuses with lysosomes, leading to degradation (Eskelinen et al., 2009; Yang and Klionsky, 2010). The canonical UPS is an ATP-dependent degradation pathway (Huang et al., 2010). Proteins are marked for proteasomal degradation by ubiquitin, a small 8.5 kDa regulatory protein, which is added to lysine residues of the target protein. Polyubiquitination, or formation of an ubiquitin side chain, specifically on lysine residue 48 of the ubiquitin moieties, targets proteins to the 26S proteasome for degradation. The ubiquitination process involves three steps: activation of the ubiquitin molecule by E1 ubiquitin enzymes, conjugation of ubiquitin to an E2 ubiquitin ligase, and ligation of the ubiquitin molecule to substrate. E3 ubiquitin ligases play a particularly important role as they connote substrate specificity and facilitate the ligation of the ubiquitin molecule to the target protein (Pickart et al., 2004).

Transient expression of high levels of Zscan4 (Zalzman et al., 2010) leads to drastic changes in stem cell properties and potency (Akiyama et al., 2015; Amano et al., 2013). Therefore, stringent regulation of the ZSCAN4 protein is required to effectively control its cellular functions. However, the regulation of human ZSCAN4 protein, and more specifically its turnover dynamics, remains obscure. As a growing body of evidence suggests a significant role for ZSCAN4 in stem cells and cancer, knowledge of its protein regulation is critical. In this study, we demonstrate for the first time that ZSCAN4 protein degradation is regulated by the ubiquitin-proteasome system.

Examples of Materials and Methods

Cell Culture

Tu167 cells were obtained from the University of Texas MD Anderson Cancer Center (Houston, Tex., USA). All cell lines used in this study were grown in complete DMEM medium (Invitrogen) supplemented with 10% fetal bovine serum (Sigma), 2 mM GlutaMAX, penicillin (100 U/mL), streptomycin (100 μg/mL) and were tested free of Mycoplasma.

Determination of ZSCAN4 Half-Life

WT and doxycycline (Dox) inducible tet-ZSCAN4 Tu167 cells were treated with 1 μg/mL Dox (or kept untreated) for 24 h to induce ZSCAN4. Cells were treated with 25 μg/ml CHX (Sigma), for the indicated time points. Total cell lysate in RIPA buffer was loaded on 10% SDS PAGE gel and immunoblotted with ZSCAN4 antibodies (1:1000; Origene;) or controls, β-actin (1:1000; Millipore) and Lamin B antibodies (1:2000; Santa Cruz). Band intensities of ZSCAN4 were quantified using ImageJ software (Schneider et al., 2012) and normalized to controls. The relative levels of ZSCAN4 in sample not treated with CHX was considered as initial level of ZSCAN4 and considered as 1 unit. The half-life of ZSCAN4 was determined using formula t½=ln 2/k (k is the slope of the degradation curve).

Autophagy Pathway Assay

tet-ZSCAN4 Tu167 cells were treated for 24 h with Dox and then autophagy inhibitors: 5 nM of Bafilomycin A1 or 25 μM of Chloroquine (Sigma) were added for 24 h. Whole cell lysate (50 μg) in RIPA buffer was used on 8% SDS-PAGE analyzed by immunoblot to visualize the following antigens: anti-ZSCAN4 (1:1000; Origene), anti-p62 (1:5000; Sigma), anti-LC3 (1:1000; CellSignaling Technology), Anti-Beta Actin (1:10,000; Millipore). All data shown represent at least 3 independent experiments.

Immunoblot Analysis

Nuclear proteins were fractionated using Nuclear Extraction Kit following manufacture's protocol (Active Motif). Total cell lysate was prepared in RIPA buffer and sonicated. For the detection of endogenous ZSCAN4 in Tu167 cells, cells were harvested by accutase (Millipore) and Cytoskeleton buffer (10 mM PIPES, 300 mM sucrose, 100 mM NaCl, 3 mM MgCl₂, 1 mM EGTA and 0.5% Triton X100) was used to fractionate cytosolic proteins. Then, pellets were lysed in urea solution (8 M Urea in 0.01 Tris pH 8+0.1 M NaH₂PO₄) and sonicated. Nuclear proteins were electrophoresed in 8% polyacrylamide gels and transferred to a PVDF membrane. Immunoblot was performed using the following primary antibodies: ZSCAN4 (Origene; 1:1000), GAPDH (Santa Cruz; 1:5000), Actin (Sigma; 1:500), Lamin B (Santa Cruz; 1:2000) and with HRP (horseradish peroxidase) conjugated secondary antibodies (Millipore; 1:5000). Protein bands were detected using Pierce ECL Western Blotting Substrate (Thermo Scientific). SuperSignal West Femto (Thermo Scientific) was used to detect endogenous ZSCAN4 in Tu167 cells. All immunoblots shown represent at least 3 independent experiments.

Ubiquitination Assay

WT Tu167 cells were treated with indicated concentration of MG132 for 3-12 h. Cells treated with vehicle only were used as controls. Nuclear protein was isolated using urea extraction buffer. Then, 100 μg of nuclear lysate was diluted in IP-RIPA buffer with 0.1% SDS and denatured by heating to 90° C. for 10 minutes. Samples were taken for co-immunoprecipitation and immunoblot analyses.

Co-Immunoprecipitation

WT Tu167 were lysed in RIPA buffer and sonicated. Protein A/G beads (Invitrogen) were incubated with 1 μg of anti-ZSCAN4 in 100 uL of RIPA buffer without SDS for 1 h at room temperature. Then, the beads were washed twice with RIPA buffer and crosslinked to the beads with 5 mM BS3 solution (ThermoFisher Scientific) according to manufacturer's protocols. Then the beads were pre-cleared with 100 μg of nuclear lysate in 100 μl IP buffer without SDS (Cell Signaling Technology) overnight at 4° C. Cell lysates were loaded and bound antigens were eluted with 25 μL RIPA with 0.1% SDS and 25 μL of 2× loading dye. Proteins were separated in 10% SDS PAGE and immunoblotted with corresponding antibodies.

Immunofluorescence Confocal Microscopy

Cells were fixed in ice cold methanol/acetic acid (3:1). The fixed cells were dropped on microscope slides. Antigen retrieval was performed followed by blocking for 10 min at room temperature. Primary antibody was incubated overnight at 4° C. for the following antigens: ZSCAN4 (Origene, 1:1000) and Ubiquitin Lysine 48 (Millipore 1: 2000). The slides were washed and incubated with secondary antibodies conjugated with Alexa-488 or 568 at room temperature for 1 h and then treated with DAPI and To-Pro-3 to stain the nuclei. Slides were mounted and visualized by a Zeiss 510-confocal microscope. Co-localization analyses were performed by ImageJ software (Schneider et al., 2012);

Quantitative reverse transcriptase polymerase chain reaction (qRT-PCR). RNA was isolated by Trizol (invitrogene) and 1 μg of total RNA was reverse transcribed by Superscript III (Invitrogen) following the manufacturer's protocol. Then, 10 ng cDNA was used per well in triplicates using SYBR green (Roche) following the manufacturer's protocol. Real time PCR was performed on LightCycler 480 (Roche). Fold induction was calculated by the absolute quantification method. Primers used:

ZSCAN4 forward
(SEQ ID NO: 22)
5′-ATCCACCTGCCTTAGTCCAC-3′
and
ZSCAN4 reverse
(SEQ ID NO: 23)
5′-TCGAAGAACTGTTCCAGCCA-3′,
and
RPLP0 reverse
(SEQ ID NO: 24)
5′-CCCATTCTATCATCAACGGGTACAA-3′.

Statistical analyses. All data and representative images shown are the result of at least three independent experiments, with biological replicates. Student's t-test, one-way or two-way ANOVAs with repeated measures were followed by Tukey's posthoc comparison tests (when appropriate) for statistical analyses. Statistical analyses were performed with STATISTICA-12 and GraphPad-Prism7 software. All P values <0.05 were considered as statistically significant.

Examples of Results

ZSCAN4 Protein Turnover and Half-Life

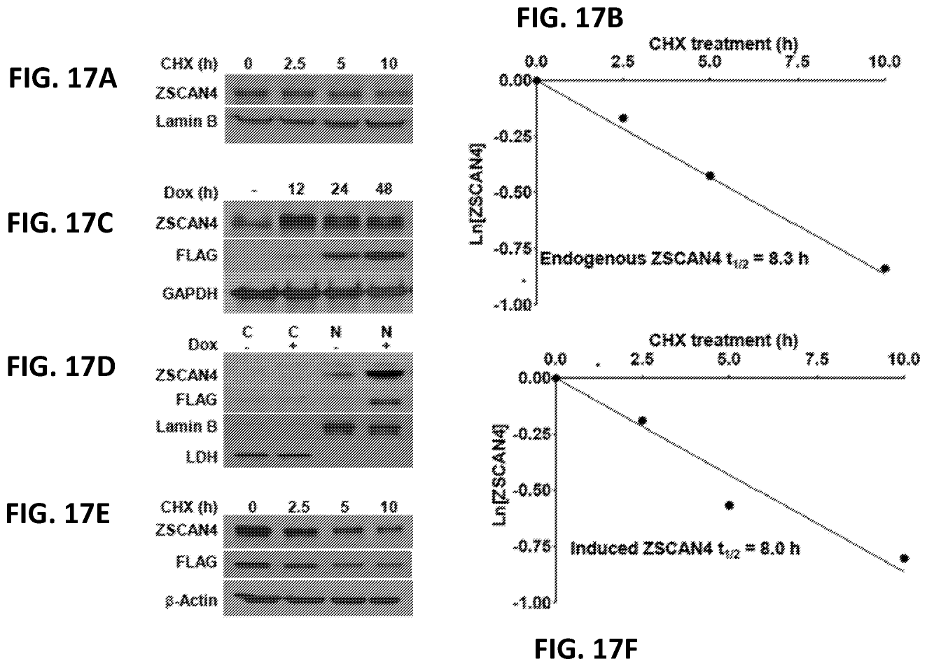

Zscan4 expression is transient and limited to a small fraction of cells, suggesting it requires tight regulation at the protein level. Interestingly, ZSCAN4 has been found to be relatively stable in mouse ES cells, with a half-life of up to 6 h (Storm et al., 2014). While normal physiological expression is limited to early stage embryonic cells, ZSCAN4 has been shown to be aberrantly reactivated in cancer (Lee et al., 2014). Therefore, ZSCAN4 protein stability was chosen to study in the human head and neck cancer cell line Tu167. To determine ZSCAN4 half-life, Tu167 cells were incubated with the protein translation inhibitor cycloheximide (CHX) and ZSCAN4 concentration was monitored by immunoblot over 10 h. The CHX chase assay indicates that ZSCAN4 protein half-life is 8.3 h (FIGS. 17A, 17B).

ZSCAN4 is weakly expressed in the human cell line Tu167. Therefore, in order to further study ZSCAN4 in these cells, a doxycycline (Dox) inducible ZSCAN4 lentiviral vector was developed. This vector was used to generate inducible ZSCAN4 cells (Tu167) and they were named tet-ZSCAN4. Addition of Dox, a tetracycline analog, to the media results in ZSCAN4 induction. The immunoblot analysis reveals that ZSCAN4 is induced in these cells as early as 12 h (FIG. 17C) and as expected, is localized to the nucleus (FIG. 17D). Using the tet-ZSCAN4 cell line, the stability of the ZSCAN4 protein was analyzed. Cells were treated for 24 h with Dox and then incubated with CHX. ZSCAN4 concentration was again monitored by immunoblot over 10 h. The CHX chase assay indicates that the induced ectopic ZSCAN4 protein half-life of 8.0 h is similar to the endogenous ZSCAN4, suggesting its utility as a tool to further study ZSCAN4 stability (FIG. 17E, 17F). These data together suggest that the stability of the human ZSCAN4 in human cancer cells is similar to that of mouse ES cells (Storm et al., 2014).

ZSCAN4 Degradation is Proteasome Dependent

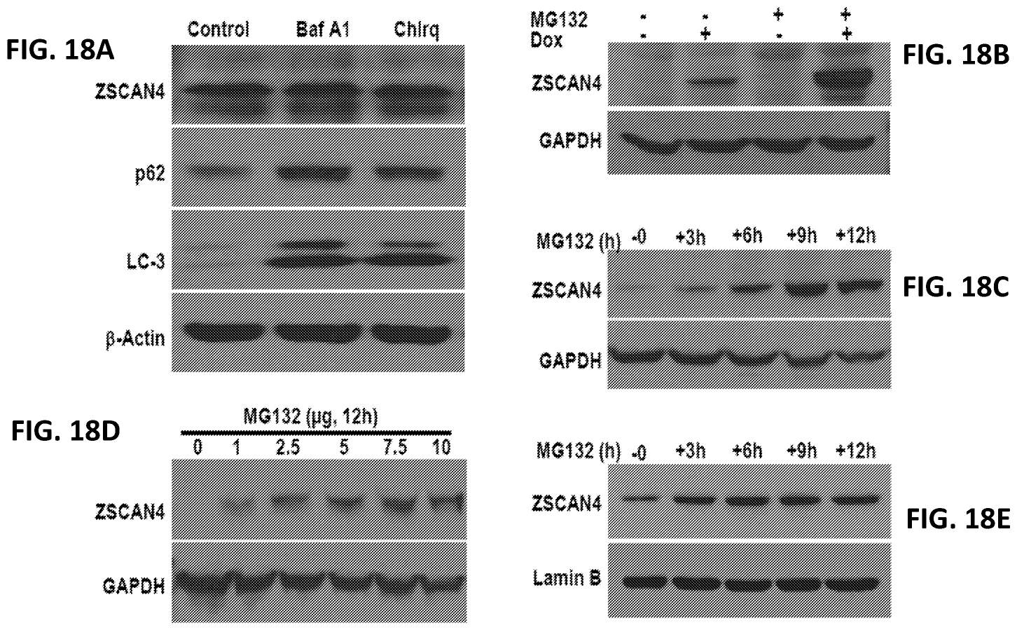

The pathway responsible for ZSCAN4 turnover and degradation was considered. The possible clearance of ZSCAN4 by autophagy was first considered. tet-ZSCAN4 cells (Tu167) were incubated for 12 h with autophagy inhibitors Bafilomycin A1 and Chloroquine (Eskelinen et al., 2009). The immunoblot analysis shows that both p62 and LC-3, two proteins specifically degraded by autophagy, accumulate in the cells (FIG. 18A). These results indicate that the autophagy pathway was successfully inhibited. However, ZSCAN4 levels remained unchanged (FIG. 18A), excluding autophagy as the mechanism responsible for ZSCAN4 turnover.

This prompted examination of the UPS. tet-ZSCAN4 cells were incubated with the proteasome inhibitor MG132 at multiple time points. The data indicate a time dependent accumulation of ZSCAN4 through the 12 h incubation (FIG. 18B). Furthermore, we show a time dependence (FIG. 18C) and a dose response (FIG. 18D) to proteasome inhibition by MG132. These data show that UPS inhibition results in ZSCAN4 accumulation and suggests it is the pathway by which ZSCAN4 protein is degraded.

To exclude that the response was specific to the exogenous ZSCAN4 induction, wild type (WT) Tu167 cells were treated with 5 uM MG132 for 0-12 h. The results validate that endogenous ZSCAN4 accumulates with proteasome inhibition (FIG. 18E). These data indicate that both the endogenous and the induced ZSCAN4 proteins are not degraded by autophagy but instead in a proteasome dependent manner.

ZSCAN4 is Marked for Proteasomal Degradation by Lysine 48 Ubiquitination

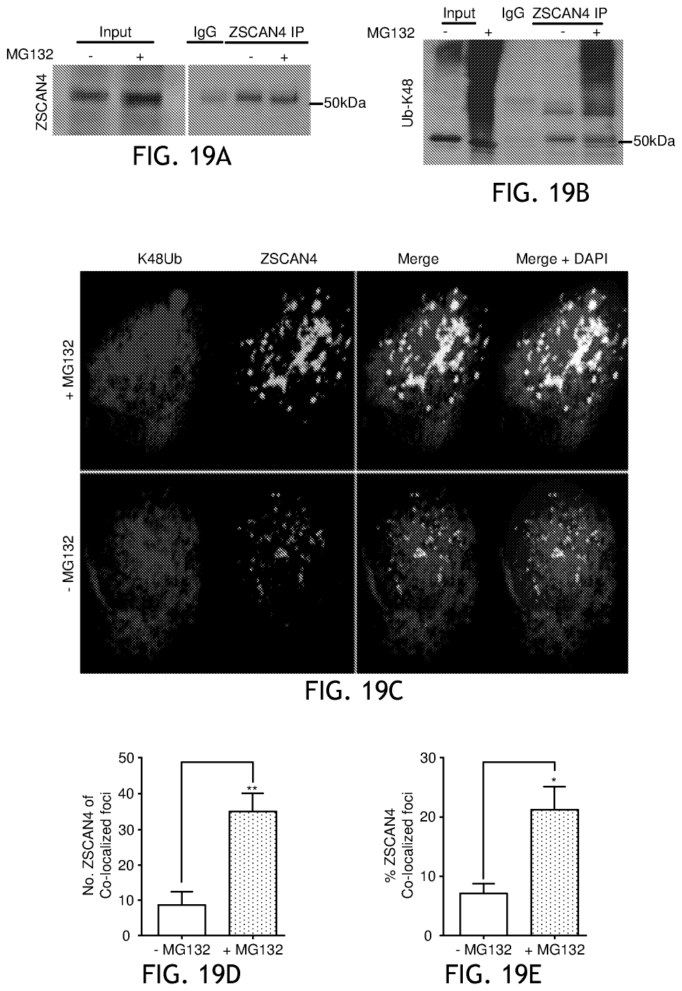

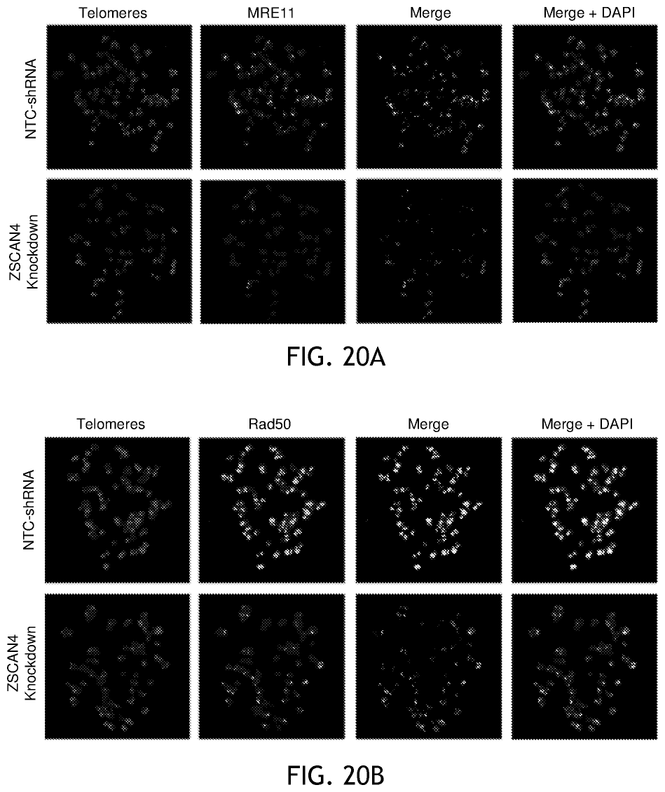

Proteins turnover allows for clearance of non-functional proteins. Proteins destined for degradation by the proteasome are tagged by ubiquitin side chains (Li et al., 2008). Lysine 48 ubiquitin side chains, also known as K48 polyubiquitination, connote specificity to proteasomal degradation (Pickart et al., 2004). Therefore, it was examined if ZSCAN4 is lysine 48 polyubiquitinated by inhibiting the proteasome with MG132 for 12 h in WT Tu167 cells. Then, covalently bound K48 ubiquitin chains were selected for by performing ZSCAN4 IP experiments in denaturing conditions. Following ZSCAN4 IP, the inventors immunoblotted with a lysine 48 ubiquitin (K48-Ub) antibody and observed a strong ubiquitin signal at the 50 kDa molecular weight of ZSCAN4 (FIG. 19A). Moreover, a significant smear above the 50 kDA band indicates ZSCAN4 with bound ubiquitin side chains of varying lengths (FIG. 19B).

To further validate the lysine 48 polyubiquitination of ZSCAN4 protein, WT Tu167 cells were treated with or without MG132 for 5 h and co-immunostained for anti-K48-Ub and anti-ZSCAN4 (FIG. 19C). Co-localization analysis indicates that about 5% of ZSCAN4 co-localizes with K48-Ub in the untreated conditions, whereas this fraction is 4 fold higher following 5 h inhibition of the proteasome with MG132 (FIG. 19D, 19E). The data suggest that ZSCAN4 is targeted to the proteasome via canonical lysine 48 polyubiquitination.

### Example 3

### Zscan4 Depletion Sequesters MRE11 and RAD50 from the Telomeres

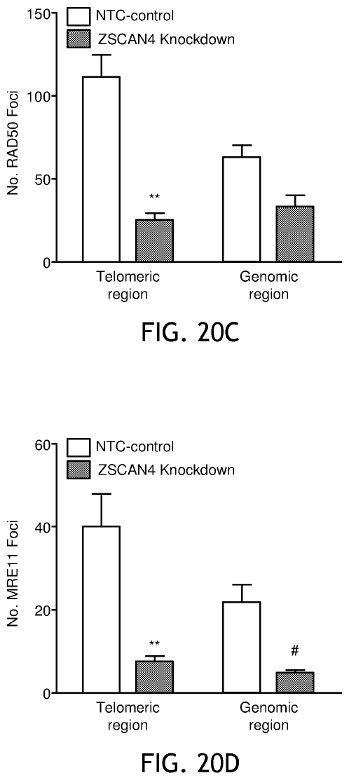

RAD50 and MRE11A, while having known function at the telomeres, also affect DNA repair globally (Czornak et al., 2008). The interaction of ZSCAN4 with MRE11 and RAD50 has prompted study if these interactions are telomere specific, or more broadly associated with genomic DNA. To investigate this, ZSCAN4 knockdown Tu167 cell lines were used along with scrambled non-targeting control shRNA (NTC-shRNA) cell lines. These cells were then analyzed with immunostaining combined with Telomere Fluorescence In Situ Hybridization (T-FISH) analysis. First, the data confirmed previous reports (Czornak et al., 2008; Zhong et al., 2007; Lamarche et al., 2010; Tsukamoto et al., 2001; Jiang et al., 2005) that MRE11 and RAD50 interact with the telomere regions (FIGS. 20A, 20B). More importantly, the data confirm that loss of ZSCAN4 leads to a significant 3-fold reduction in the average number of RAD50 foci (FIG. 20C) and a 5.4-fold decrease in the average number of MRE11 foci at telomeres (FIG. 20D). This was determined based on localization of each protein of interest with the ends of the chromosomes. Furthermore, the data indicate that the major effect of ZSCAN4 depletion was on telomeric RAD50 and MRE11 foci. No significant effect was observed on the genomic foci of RAD50 and only a marginal effect was detected on MRE11 genomic foci (P≤0.054) (FIGS. 20C, 20D). Taken together, this indicates that at least in certain embodiments ZSCAN4 is required for MRE11 and RAD50 localization to the telomeres.

### Example 4

### Inhibition of MRE11 Catalytic Activity Reduces Zscan4-Mediated Telomere Regulation

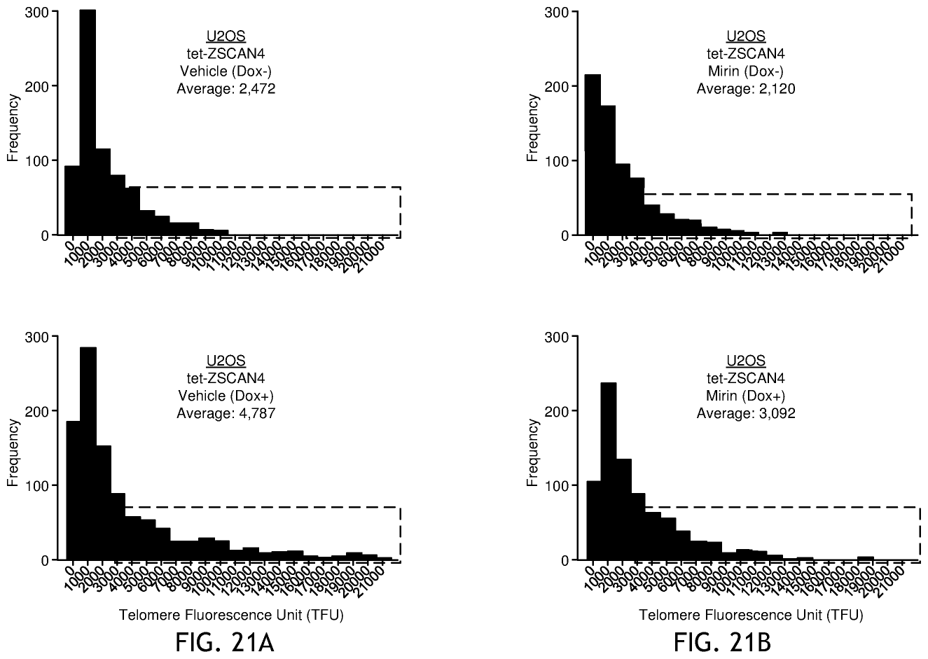

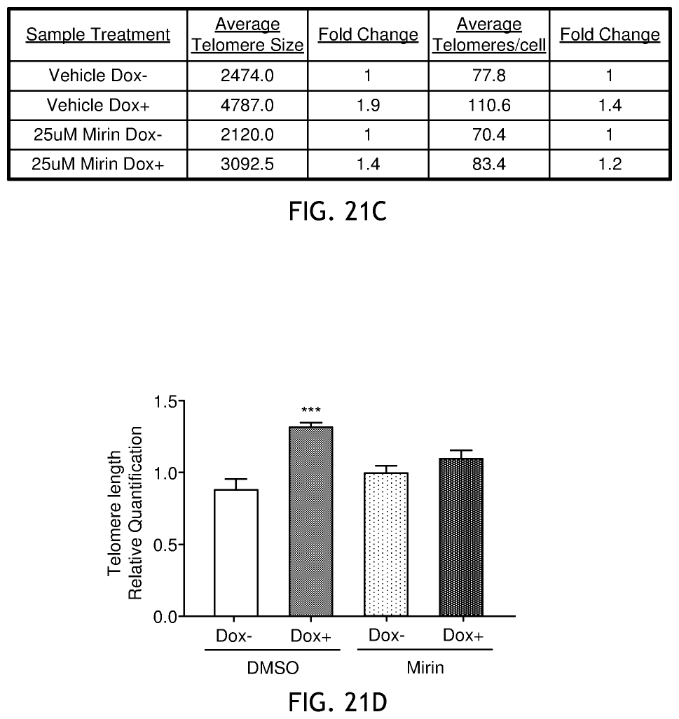

MRE11 has been shown to have multiple catalytic functions in telomeric maintenance and DNA damage repair (Attwooll et al., 2009; Deng et al., 2009). Of note is the 3′-5′ exonuclease activity involved in strand resection, presumably to prepare single strand DNA for strand invasion required for recombination (Paull and Gellert, 1998). In order to define the significance of MRE11 to ZSCAN4 function we performed an experiment to block MRE11 in ZSCAN4 inducible cells and study the effect on telomere extension (Dupré et al., 2008). This was accomplished with the MRE11 specific inhibitor, Mirin, which selectively binds and inhibits its exonuclease activity. To study the effect of Mirin, ZSCAN4 was induced by Dox, as we described above, and measured telomere length via Quantitative Fluorescence In Situ Hybridization (Q-FISH). As expected, following ZSCAN4 induction in the vehicle treated (DMSO only) controls we show a 1.9-fold increase in telomere length, which corroborates our previous findings (FIG. 21A). Conversely, ZSCAN4 induction following Mirin treatment has a significantly reduced effect on telomere extension (FIG. 21B). Further, the quantity of detectable telomeres per nuclei increases in the ZSCAN4 induced, DMSO vehicle treated samples while samples treated with MRE11 inhibitor Mirin have significantly fewer detectable telomeres (FIG. 21C). The effect of Mirin on ZSCAN4 mediated telomere extension was confirmed via telomere length qPCR assay in tet-FLAG-ZSCAN4 cells (FIG. 21D). Taken together, these results indicate that in certain embodiments ZSCAN4 requires the catalytic activity of MRE11 to effectively extend telomeres in cancer.

The scope of the present application is not intended to be limited to the particular embodiments of the process, machine, manufacture, composition of matter, means, methods and steps described in the specification.

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
Akiyama, T., L. Xin, M. Oda, A. A. Sharov, M. Amano, Y. Piao, J. S. Cadet, D. B. Dudekula, Y. Qian, W. Wang, S. B. Ko, M. S. Ko, Transient bursts of Zscan4 expression are accompanied by the rapid derepression of heterochromatin in mouse embryonic stem cells, DNA research: an international journal for rapid publication of reports on genes and genomes, (2015).
Amano, T., T. Hirata, G. Falco, M. Monti, L. V. Sharova, M. Amano, S. Sheer, H. G. Hoang, Y. Piao, C. A. Stagg, K. Yamamizu, T. Akiyama, M. S. Ko, Zscan4 restores the developmental potency of embryonic stem cells, Nature communications, 4 (2013) 1966.
Arora, R., Brun, C. M. & Azzalin, C. M. Transcription regulates telomere dynamics in human cancer cells. RNA 18, 684-93 (2012).
Arora, R., Lee, Y., Wischnewski, H. & Brun, C. M. RNaseH1 regulates TERRA-telomeric DNA hybrids and telomere maintenance in ALT tumour cells. Nature . . . (2014).
Attwooll, C. L., nar, M. & Petrini, J. H. J. The mre11 complex and the response to dysfunctional telomeres. Molecular and cellular biology 29, 5540-5551 (2009).
Azzalin, C. M., Reichenbach, P., Khoriauli, L., Giulotto, E. & Lingner, J. Telomeric repeat containing RNA and RNA surveillance factors at mammalian chromosome ends. Science (New York, N.Y.) 318, 798-801 (2007).
Bailey, S. M., Goodwin, E. H., Meyne, J. & Cornforth, M. N. CO-FISH reveals inversions associated with isochromosome formation. Mutagenesis 11, 139-44 (1996).
Balk, B. et al. Telomeric RNA-DNA hybrids affect telomere-length dynamics and senescence. Nature structural & molecular biology 20, 1199-1205 (2013).
Boguslawski, S. J. et al. Characterization of monoclonal antibody to DNA.RNA and its application to immunodetection of hybrids. J Immunol Methods 89, 123-30 (1986).
Bryan, T. M., Englezou, A., Dalla-Pozza, L., Dunham, M. A. & Reddel, R. R. Evidence for an alternative mechanism for maintaining telomere length in human tumors and tumor-derived cell lines. Nat Med 3, 1271-4 (1997).
Callicott, R. J. & Womack, J. E. Real-time PCR assay for measurement of mouse telomeres. Comp Med 56, 17-22 (2006).
Cawthon, R. M. Telomere measurement by quantitative PCR. Nucleic Acids Res 30, e47 (2002).
Ciechanover, A. Proteolysis: from the lysosome to ubiquitin and the proteasome, Nature reviews. Molecular cell biology, 6 (2005) 79-87.
Compendia Bioscience, A. A., MI. Oncomine. https://www.oncomine.org/resource/login.html. (2015).
Czornak, K., S. Chughtai, and K. H. Chrzanowska, Mystery of DNA repair: the role of the MRN complex and ATM kinase in DNA damage repair. Journal of Applied Genetics, 2008. 49(4): p. 383-396.
Deng, Y., Guo, X., Ferguson, D. O. & Chang, S. Multiple roles for MRE11 at uncapped telomeres. Nature 460, 914-8 (2009).
Deng, Z., Norseen, J., Wiedmer, A., Riethman, H. & Lieberman, P. M. TERRA RNA binding to TRF2 facilitates heterochromatin formation and ORC recruitment at telomeres. Molecular cell 35, 403-413 (2009).
Dewar, J. M. & Lydall, D. Telomere replication: Mre11 leads the way. Mol Cell 38, 777-9 (2010).
Dupré, A., et al., A forward chemical genetic screen reveals an inhibitor of the Mre11-Rad50-Nbs1 complex. Nature chemical biology, 2008. 4(2): p. 119-125.
Edelstein, L. C. & Collins, T. The SCAN domain family of zinc finger transcription factors. Gene 359, 1-17 (2005).
Eskelinen, E. L., P. Saftig, Autophagy: a lysosomal degradation pathway with a central role in health and disease, Biochimica et biophysica acta, 1793 (2009) 664-673.
Falco, G., S. L. Lee, I. Stanghellini, U. C. Bassey, T. Hamatani, M. S. Ko, Zscan4: a novel gene expressed exclusively in late 2-cell embryos and embryonic stem cells, Developmental biology, 307 (2007) 539-550.
Forbes, S. A. et al. COSMIC: exploring the world's knowledge of somatic mutations in human cancer. Nucleic Acids Res 43, D805-11 (2015).
Gilley, D., Tanaka, H. & Herbert, B. S. Telomere dysfunction in aging and cancer. Int J Biochem Cell Biol 37, 1000-13 (2005).
Goodwin, E. & Meyne, J. Strand-specific FISH reveals orientation of chromosome 18 alphoid DNA. Cytogenet Cell Genet 63, 126-7 (1993).
Haber, J. E. The many interfaces of Mre11. Cell 95, 583-6 (1998).
Hayflick, L., Moorhead, P. S. The serial cultivation of human diploid cell strains. Exp Cell Res 25, 585-621 (1961).
Hirata, T., T. Amano, Y. Nakatake, M. Amano, Y. Piao, H. G. Hoang, M. S. Ko, Zscan4 transiently reactivates early embryonic genes during the generation of induced pluripotent stem cells, Sci Rep, 2 (2012) 208.
Hu, J. et al. Antitelomerase therapy provokes ALT and mitochondrial adaptive mechanisms in cancer. Cell 148, 651-63 (2012).
Hu, Z., Zhang, A., Storz, G., Gottesman, S. & Leppla, S. H. An antibody-based microarray assay for small RNA detection. Nucleic Acids Res 34, e52 (2006).
Huang, J. et al. Association of telomere length with authentic pluripotency of ES/iPS cells. Cell Res 21, 779-92 (2011).
Huang, Q., M. E. Figueiredo-Pereira, Ubiquitin/proteasome pathway impairment in neurodegeneration: therapeutic implications, Apoptosis: an international journal on programmed cell death, 15 (2010) 1292-1311.
Jiang, J., W. Lv, X. Ye, L. Wang, M. Zhang, H. Yang, M. Okuka, C. Zhou, X. Zhang, L. Liu, J. Li, Zscan4 promotes genomic stability during reprogramming and dramatically improves the quality of iPS cells as demonstrated by tetraploid complementation, Cell research, 23 (2013) 92-106.
Jiang, W.-Q., et al., Suppression of Alternative Lengthening of Telomeres by Sp100-Mediated Sequestration of the MRE11/RAD50/NBS1 Complex. Molecular and cellular biology, 2005. 25(7): p. 2708-2721.
Joseph, I. S. et al. An mre11 mutation that promotes telomere recombination and an efficient bypass of senescence. Genetics 185, 761-70 (2010).
Korolchuk, V. I., F. M. Menzies, D. C. Rubinsztein, Mechanisms of cross-talk between the ubiquitin-proteasome and autophagy-lysosome systems, FEBS letters, 584 (2010) 1393-1398.
Kunická, Z., Mucha, I. & Fajkus, J. Telomerase activity in head and neck cancer. Anticancer Res 28, 3125-9 (2008).
Lamarche, B. J., N. I. Orazio, and M. D. Weitzman, The MRN complex in Double-Strand Break Repair and Telomere Maintenance. FEBS letters, 2010. 584(17): p. 3682-3695.
Laud, P. R. et al. Elevated telomere-telomere recombination in WRN-deficient, telomere dysfunctional cells promotes escape from senescence and engagement of the ALT pathway. Genes Dev 19, 2560-70 (2005).
Lechel, A. et al. The cellular level of telomere dysfunction determines induction of senescence or apoptosis in vivo. EMBO Rep 6, 275-81 (2005).
Lee, J. H. et al. Regulation of Mre11/Rad50 by Nbs1: effects on nucleotide-dependent DNA binding and association with ataxia-telangiectasia-like disorder mutant complexes. J Biol Chem 278, 45171-81 (2003).
Lee, K., L. S. Gollahon, ZSCAN4 and TRF1: A functionally indirect interaction in cancer cells independent of telomerase activity, Biochem Biophys Res Commun, 466 (2015) 644-649.
Lee, K., L. S. Gollahon, Zscan4 interacts directly with human Rap1 in cancer cells regardless of telomerase status, Cancer biology & therapy, 15 (2014) 1094-1105.
Li, W., Y. Ye, Polyubiquitin chains: functions, structures, and mechanisms, Cellular and molecular life sciences: CMLS, 65 (2008) 2397-2406.
Liu, T. J. et al. Apoptosis induction by E2F-1 via adenoviral-mediated gene transfer results in growth suppression of head and neck squamous cell carcinoma cell lines. Cancer Gene Ther 6, 163-71 (1999).
Nakai-Futatsugi, Y. & Niwa, H. Zscan4 Is Activated after Telomere Shortening in Mouse Embryonic Stem Cells. Stem Cell Reports 6, 483-95 (2016).
Nandi, D., P. Tahiliani, A. Kumar, D. Chandu, The ubiquitin-proteasome system, Journal of biosciences, 31 (2006) 137-155.
Ohbo, K. et al. Modulation of hematopoiesis in mice with a truncated mutant of the interleukin-2 receptor gamma chain. Blood 87, 956-67 (1996).
Park, H. S., I. Hwang, K. A. Choi, H. Jeong, J. Y. Lee, S. Hong, Generation of induced pluripotent stem cells without genetic defects by small molecules, Biomaterials, 39 (2015) 47-58.
Paull, T. T. and M. Gellert, The 3′ to 5′ Exonuclease Activity of Mre11 Facilitates Repair of DNA Double-Strand Breaks. Molecular cell, 1998. 1(7): p. 969-979.
Pavri, R., B. Zhu, G. Li, P. Trojer, S. Mandal, A. Shilatifard, D. Reinberg, Histone H2B monoubiquitination functions cooperatively with FACT to regulate elongation by RNA polymerase II, Cell, 125 (2006) 703-717.
Pickart, C. M., M. J. Eddins, Ubiquitin: structures, functions, mechanisms, Biochimica et biophysica acta, 1695 (2004) 55-72.
Poon, S. S., Martens, U. M., Ward, R. K. & Lansdorp, P. M. Telomere length measurements using digital fluorescence microscopy. Cytometry 36, 267-78 (1999).
Porro, A., Feuerhahn, S. & Lingner, J. TERRA-reinforced association of LSD1 with MRE11 promotes processing of uncapped telomeres. Cell reports 6, 765-776 (2014).
Ren, P., Z. Sheng, Y. Wang, X. Yi, Q. Zhou, J. Zhou, S. Xiang, X. Hu, J. Zhang, RNF20 promotes the polyubiquitination and proteasome-dependent degradation of AP-2alpha protein, Acta biochimica et biophysica Sinica, 46 (2014) 136-140.
Rhodes, D. R. et al. ONCOMINE: a cancer microarray database and integrated data-mining platform. Neoplasia 6, 1-6 (2004).
Scheibe, M., Arnoult, N., Kappei, D. & Buchholz, F. Quantitative interaction screen of telomeric repeat-containing RNA reveals novel TERRA regulators. Genome . . . (2013).
Schneider, C. A., Rasband, W. S. & Eliceiri, K. W. NIH Image to ImageJ: 25 years of image analysis. Nat Methods 9, 671-5 (2012).
Schoeftner, S. & Blasco, M. A. Developmentally regulated transcription of mammalian telomeres by DNA-dependent RNA polymerase II. Nat Cell Biol 10, 228-36 (2008).
Shema, E., I. Tirosh, Y. Aylon, J. Huang, C. Ye, N. Moskovits, N. Raver-Shapira, N. Minsky, J. Pirngruber, G. Tarcic, P. Hublarova, L. Moyal, M. Gana-Weisz, Y. Shiloh, Y. Yarden, S. A. Johnsen, B. Vojtesek, S. L. Berger, M. Oren, The histone H2B-specific ubiquitin ligase RNF20/hBRE1 acts as a putative tumor suppressor through selective regulation of gene expression, Genes Dev, 22 (2008) 2664-2676.
Storm, M. P., B. Kumpfmueller, H. K. Bone, M. Buchholz, Y. Sanchez Ripoll, J. B. Chaudhuri, H. Niwa, D. Tosh, M. J. Welham, Zscan4 is regulated by PI3-kinase and DNA-damaging agents and directly interacts with the transcriptional repressors LSD1 and CtBP2 in mouse embryonic stem cells, PloS one, 9 (2014) e89821.
Tsukamoto, Y., A. K. P. Taggart, and V. A. Zakian, The role of the Mre11-Rad50-Xrs2 complex in telomerase-mediated lengthening of Saccharomyces cerevisiae telomeres. Current Biology, 2001. 11(17): p. 1328-1335.
Yan, L. et al. Single-cell RNA-Seq profiling of human preimplantation embryos and embryonic stem cells. Nat Struct Mol Biol 20, 1131-9 (2013).
Yang, Q. Cellular senescence, telomere recombination and maintenance. Cytogenet Genome Res 122, 211-8 (2008).
Yang, Z., D. J. Klionsky, Mammalian autophagy: core molecular machinery and signaling regulation, Current opinion in cell biology, 22 (2010) 124-131.
Yu, T.-Y. Y., Kao, Y.-w. W. & Lin, J.-J. J. Telomeric transcripts stimulate telomere recombination to suppress senescence in cells lacking telomerase. Proceedings of the National Academy of Sciences of the United States of America 111, 3377-3382 (2014).
Zalzman, M., G. Falco, L. V. Sharova, A. Nishiyama, M. Thomas, S. L. Lee, C. A. Stagg, H. G. Hoang, H. T. Yang, F. E. Indig, R. P. Wersto, M. S. H. Ko, Zscan4 regulates telomere elongation and genomic stability in ES cells, Nature, 464 (2010) 858-U866.
Zalzman, M. et al. Zscan4 regulates telomere elongation and genomic stability in ES cells. Nature 464, 858-63 (2010).
Zhong, Z.-H., et al., Disruption of Telomere Maintenance by Depletion of the MRE11/RAD50/NBS1 Complex in Cells That Use Alternative Lengthening of Telomeres. Journal of Biological Chemistry, 2007. 282(40): p. 29314-29322.
Zhu, B., Y. Zheng, A. D. Pham, S. S. Mandal, H. Erdjument-Bromage, P. Tempst, D. Reinberg, Monoubiquitination of human histone H2B: the factors involved and their roles in HOX gene regulation, Mol Cell, 20 (2005) 601-611.

Although the present disclosure and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

1. A method of treating or preventing cancer in an individual, comprising the step of providing an effective amount of one or more zinc finger and SCAN domain containing 4 (ZSCAN4)-inhibiting agents that reduce the activity and/or amount of ZSCAN4 in cells of the individual and/or one or more MRE11 inhibitors.

2. The method of claim 1, wherein the ZSCAN4-inhibiting agent comprises a nucleic acid, peptide, protein, small molecule, or combination thereof.

3. The method of claim 1, wherein the ZSCAN4-inhibiting agent comprises an RNA interference nucleic acid or an antibody.

4. The method of claim 1, wherein the individual has an epithelial cancer.

5. The method of claim 1, wherein the cancer is a brain, head and neck, thyroid, breast, skin, prostate, pancreatic, lung, cervical, liver, stomach, kidney, blood, colon, lung, gastrointestinal tract, genitourinary tract, pancreatic, ovarian, testicular, urethra, bone, neuronal, gynecological hematologic, adrenal gland cancer or a benign tumor.

6. The method of claim 1, wherein the individual is at risk for the cancer.

7. The method of claim 1, wherein the individual is provided an additional cancer therapy.

8. The method of claim 7, wherein the additional cancer therapy comprises surgery, radiation, chemotherapy, immunotherapy, hormone therapy, or a combination thereof.

9. The method of claim 7, wherein the additional cancer therapy comprises one or more G-quadruplex ligands, one or more G-quadruplex binding proteins, one or more telomerase inhibitors, one or more inhibitors of MRE11, one or more inhibitors of RAD50, or a combination thereof.

10. The method of claim 9, wherein the G-quadruplex ligand is N,N′-bis[2-1(-piperidino)-ethyl]-3,4,9,10-perylene-tetracarboxylic diimide (PIPER).

11. The method of claim 9, wherein the telomerase inhibitor is telomerase inhibitor IX, Imetelstat (GRN163L), BIBR 1532, or a combination thereof.

12. The method of claim 9, wherein the inhibitor of MRE11 and/or RAD50 comprises a nucleic acid, peptide, protein, small molecule, or combination thereof.

13. The method of claim 9, wherein the MRE11 inhibitor is Mirin [5-(4-Hydroxybenzylidene)-2-iminothiazolidin-4-one]; PFM01[(5Z)-5-[(4-Hydroxyphenyl)methylene]-3-(2-methylpropyl)-2-thioxo-4-thiazolidinone, 5-(4-Hydroxybenzylidene)-3-isobutyl-2-thioxothiazolidin-4-one]; or a combination thereof.

14. The method of claim 1, wherein the cancer is resistant to one or more chemotherapies.

15. The method of claim 1, wherein the cancer is at risk for becoming resistant to one or more chemotherapies.

16. The method of claim 1, wherein the MRE11 inhibitor is Mirin or a MRE11 antibody.
